(19) United States
(12) Patent Application Publication
(10) Pub. No.: US 2022/0080140 A1
(43) Pub. Date: Mar. 17, 2022

(54) NON-INVASIVE ESTIMATION OF INTRAPLEURAL PRESSURE

(71) Applicant: Covidien LP, Mansfield, MA (US)

(72) Inventors: Kun Li, San Diego, CA (US); Richard J. Nakai, Long Beach, CA (US)

(73) Assignee: Covidien LP, Mansfield, MA (US)

(21) Appl. No.: 17/366,264

(22) Filed: Jul. 2, 2021

Related U.S. Application Data
(60) Provisional application No. 63/079,173, filed on Sep. 16, 2020.

(51) Int. Cl. A61M 16/00; G16H 20/40

(52) U.S. Cl. CPC A61M 16/026; G16H 20/40; A61M 2205/15; A61M 2016/0033; A61M 2016/0018; A61M 2016/0027

(57) ABSTRACT

Systems and methods for non-invasively estimating intrapleural pressure and muscle pressure are disclosed. The disclosed estimation of intrapleural pressure functions in the presence of a leak and/or leak-compensated flow and does not require a maneuver. In examples, the muscle pressure is represented as a muscle pressure model with model parameters that are unique from breath to breath. An equation of motion relates the muscle pressure, respiratory mechanics, and measured ventilation data (e.g., airway pressure and flow). Based on past measured ventilation data, values for the respiratory mechanics and the model parameters are estimated. Constraints may be set on these values. An intrapleural pressure profile may be generated for past inhalation phases, based on the past measured ventilation data and the estimated model parameters and/or the estimated respiratory mechanics. The estimated respiratory mechanics may be used for real-time estimates of intrapleural pressure with real-time ventilation data.

### CROSS-REFERENCE TO RELATED APPLICATIONS

This application claims the benefit of U.S. Provisional Application No. 63/079,173, filed Sep. 16, 2020, the complete disclosure of which is hereby incorporated herein by reference in its entirety.

### INTRODUCTION

Medical ventilator systems have long been used to provide ventilatory and supplemental oxygen support to patients. These ventilators typically include a connection for pressurized gas (air, oxygen) that is delivered to the patient through a conduit or tubing. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

It is with respect to this general technical environment that aspects of the present technology disclosed herein have been contemplated. Furthermore, although a general environment is discussed, it should be understood that the examples described should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for non-invasive estimation of intrapleural pressure. Aspects disclosed herein include a method for delivering ventilation. The method includes receiving a support setting identifying an amount of proportional assistance to provide to the patient. Additionally, the method includes receiving a set of measurements, the set of measurements including at least one airway pressure and at least one flow measurement. Based on the set of measurements, the method includes estimating a lung compliance and a lung resistance, without using a hold maneuver. The method also includes receiving a real-time airway pressure and a real-time flow for a current breath. Based on the real-time airway pressure, the real-time flow, the estimated lung compliance, and the estimated lung resistance, the method includes estimating at least one of a real-time intrapleural pressure or a real-time muscle pressure. The method also includes delivering ventilation to the patient based on the support setting and the at least one of the real-time intrapleural pressure or the real-time muscle pressure.

In an example, the set of measurements includes a first subset of measurements from a first inhalation phase of a first breath and a second subset of measurements from a second inhalation phase of a second breath, and the first breath and the second breath occur prior to the current breath. In another example, the estimated lung resistance and the estimated lung compliance are common to the first breath and the second breath. In a further example, the at least one flow measurement of the set of measurements is leak-compensated. In yet another example, the method further includes estimating a real-time leak flow, wherein the at least one of the real-time intrapleural pressure or the real-time muscle pressure is further based on the real-time leak flow. In still a further example, ventilation is delivered according to a pressure assist ventilation (PAV) mode. In another example, the method further includes identifying a muscle pressure model with a set of model parameters; and based on the set of measurements, estimating a set of values for the set of model parameters, wherein estimating the lung compliance and the lung resistance is further based on the set of values for the set of model parameters. In a further example, the muscle pressure model is a fourth degree Bernstein basis polynomial.

Additional aspects disclosed herein include a method for non-invasively estimating an intrapleural pressure of a patient. The method includes identifying a muscle pressure model with a set of model parameters, and receiving a set of prior measurements for at least one prior breath, the set of prior measurements including at least one airway pressure and at least one flow measurement. Based on the set of prior measurements, the method includes estimating a lung compliance, a lung resistance, and at least one set of values for the set of model parameters. Additionally, the method includes receiving a set of real-time measurements for a current breath, wherein the set of real-time measurements includes at least one airway pressure and at least one flow measurement. Based on the set of real-time measurements, the at least one set of values for the set of model parameters, the muscle pressure model, the estimated lung compliance, and the estimated lung resistance, the method also includes estimating at least one of an intrapleural pressure or a muscle pressure in real time. The method further includes delivering ventilation to the patient based on the at least one of the real-time intrapleural pressure or the real-time muscle pressure.

In an example, the lung compliance, the lung resistance, and the at least one set of values for the set of model parameters are estimated by minimizing an error between the at least one airway pressure of the set of prior measurements and a modeled airway pressure based on the at least one flow measurement of the set of prior measurements. In another example, the modeled airway pressure is P_{aw}=R*Q+E*∫Qdt+PEEP−Pₘᵤₛ. In a further example, the set of prior measurements is received for at least a first prior breath and a second prior breath, and the at least one set of values for the set of model parameters includes a first value set for the first prior breath and a second value set for the second prior breath.

In a further aspect disclosed herein, a method is provided for noninvasive estimation of intrapleural pressure. The method includes accessing a muscle pressure model, and receiving a first set of measurements for a first inhalation phase of a patient and a second set of measurements for a second inhalation phase of the patient, the first set of measurements and the second set of measurements including at least one airway pressure and at least one flow. Based on the first set of measurements and the second set of measurements, the method further includes estimating a parameter set, the parameter set including a first set of model parameters of the muscle pressure model for the first inhalation phase, a second set of model parameters of the muscle pressure model for the second inhalation phase, and a lung resistance and a lung compliance common to the first inhalation phase and the second inhalation phase. Based on the second set of measurements, the method further includes generating at least one value of at least one of an intrapleural pressure or a muscle pressure of the patient for the second inhalation phase.

In an example, the at least one value for the second inhalation phase is a maximum value during the second inhalation phase. In another example, the method further includes accessing an airway pressure model, wherein the airway pressure model relates an airway pressure, a flow, a resistance, an elastance, and the muscle pressure, wherein the at least one value is generated based on the airway pressure model. In a further example, the parameter set is estimated by minimizing an error between the airway pressure model and the at least one airway pressure for the first set of measurements and the second set of measurements. In yet another example, generating the at least one value for the second inhalation phase is further based on the lung resistance and the lung compliance common to the first inhalation phase and the second inhalation phase and the airway pressure model. In still a further example, the muscle pressure model is fourth degree with a Bernstein basis polynomial. In another example, generating the at least one value for the second inhalation phase is further based on the muscle pressure model and the second set of model parameters. In a further example, the method also includes, based on the muscle pressure model, the second set of model parameters, and the second set of measurements, generating a first profile of at least one of the intrapleural pressure or the muscle pressure for the second inhalation phase; and based on the muscle pressure model, the first set of model parameters, and the first set of measurements, generating a second profile for the at least one of the intrapleural pressure or the muscle pressure for the first inhalation phase.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example of a medical ventilator connected to a human patient.

FIG. 2 a block diagram illustrating an example of a ventilator system.

FIG. 3 is an example method for non-invasively estimating intrapleural pressure.

FIG. 4 is an example method for real-time, non-invasive estimation of intrapleural pressure.

FIG. 5 is an example method for non-invasive estimation of intrapleural pressure from breath to breath, based on measurements received from a plurality of inhalation phases.

FIG. 6 is an example method for non-invasive estimation of intrapleural pressure from breath to breath, based on measurements received from a subset of prior inhalation phases.

FIG. 7 is an example method for non-invasive estimation of intrapleural pressure, based on a moving window of prior breaths.

FIG. 8 is an example method for non-invasive estimation of intrapleural pressure as implemented with a support setting.

FIG. 9 is a graphical example of intrapleural pressure over time for a patient with acute respiratory distress syndrome (ARDS), showing a first moving breath window and a second moving breath window.

FIGS. 10A-10C show graphical examples of true muscle pressure and estimated muscle pressure.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

For the purposes of this disclosure, a “breath” refers to a single cycle of inspiration and exhalation delivered with the assistance of a ventilator. The term “breath type” refers to some specific definition or set of rules dictating how the pressure and flow of respiratory gas is controlled by the ventilator during a breath. A ventilation “mode” or ventilation “type” is a set of rules governing or controlling the delivery of multiple subsequent breaths. Modes may be mandatory, as controlled by the ventilator, or spontaneous, that allows a breath to be delivered or controlled upon detection of a patient's effort to inhale, exhale, or both. For example, a simple mandatory mode of ventilation is to deliver one breath of a specified mandatory breath type at a clinician-selected respiratory rate, f (e.g., one breath every 6 seconds). Typically, ventilators will continue to provide breaths of the specified breath type as dictated by the rules defining the mode, until the mode is changed by a clinician. For example, breath types may be mandatory mode breath types where the initiation and termination of the breath is made by the ventilator, or spontaneous mode breath types where the breath is initiated and terminated by the patient. Examples of breath types utilized in the spontaneous mode of ventilation include proportional assist (PA) breath type (otherwise referred to as proportional assist ventilation or PAV), volume support (VS) breath type, pressure support (PS) breath type, etc. Examples of mandatory breath types include a volume control breath type, a pressure control breath type, volume-targeted pressure control breath type etc.

When mechanical ventilation is used, the airway pressure shown on the ventilator display does not distinguish between the lung and chest wall components. Monitoring the intrapleural pressure in a patient under mechanical ventilation may, however, provide important information not obtainable from the airway pressure alone. For example, the intrapleural pressure provides information on patient's muscle activity during spontaneous and ventilator-assisted breaths and information of respiratory system mechanics. Moreover, the intrapleural pressure may be used to determine transpulmonary pressure, which in turn helps to assess a ventilation strategy and prevent lung injury.

Intrapleural pressure, however, is difficult to measure and generally requires invasive measurement techniques. Access to the pleural cavity of a patient to obtain a direct measurement of intrapleural pressure has disadvantages including an invasive procedure and increased risk of pneumothorax. Alternatively, esophageal pressure may be measured as a surrogate for intrapleural pressure by allowing calculation of the pressure required to distend the lung and the chest wall. Measuring the esophageal pressure, however, requires placing a delicate balloon in the esophagus with an invasive procedure involving the insertion of a balloon-tipped catheter and correct placement and inflation of the balloon. Non-invasive techniques for estimating intrapleural pressure are preferable to reduce risk of infection. Accordingly, the present technology provides for methods for non-invasively estimating intrapleural pressure.

One method of non-invasively estimating the intrapleural pressure uses a respiratory mechanics maneuver (otherwise referred to as a stabilization operation, a pause maneuver, or a plateau maneuver). The maneuver is used to estimate lung resistance, R, lung compliance, C, and lung elastance, E (where lung elastance is the inverse of lung compliance, E=1/C) (collectively referred to as “respiratory mechanics”). Based on the estimated respiratory mechanics, the intrapleural pressure may be estimated using a respiratory mechanics relationship (e.g., an equation of motion). The maneuver includes the forced imposition of a stable period at the end of an inspiratory phase so that there is no flow delivery to or from the patient's lung. The stable period of the maneuver may stabilize the pressure and flow in the patient circuit so that there is no flow into or out of the patient's lungs at the point in which the lungs have taken a breath and thus are expanded with a known volume of gas (as determined during normal operation of the ventilator as discussed above with reference to the leak-compensated inspired lung volume). In practice, the stabilization of the pressure and flow is an iterative process in which the ventilator monitors the pressure and adjusts delivered flow until the pressure and flow stabilize at the point where the pressure and flow correspond to a steady state. Various methods for stabilizing pressure and flow in a ventilation tubing system are known in the art. This maneuver, however, does not work in the presence of a leak, as pressure and flow do not stabilize.

Among other things, the systems and methods disclosed herein address these circumstances by providing a non-invasive estimation of intrapleural pressure that does not require performing a maneuver and can be calculated even during the presence of a leak. At any time, the intrapleural pressure (Pₚₗ) depends on the pressure generated by all of the respiratory muscles (Pₘᵤₛ) and the pressure gradient over the chest wall (P_{cw}): Pₚₗ=Pₘᵤₛ+P_{cw}. The pressure gradient over the chest wall, P_{cw}, may be calculated by dividing the inspired volume by the chest wall compliance (C_{cw}), which may be estimated as 4% of vital capacity. The muscle pressure, which is the pressure generated by the respiratory muscles in the thoracic cavity (including the left pleural cavity, the right pleural cavity, and the mediastinum), may be represented as a muscle pressure model over an inhalation phase of a breath. The muscle pressure model includes one or more model parameters that are unique from breath to breath. The respiratory mechanics and the model parameters for each breath (as used to estimate the muscle pressure using the muscle pressure model), are based on measurements received during the inhalation phase of one or more prior breaths. To estimate the respiratory mechanics and the model parameters for the muscle pressure model an error may be minimized to provide estimates of their values. An intrapleural pressure profile may be generated for past inhalation phases, based on the estimated model parameters or based on the estimated respiratory mechanics and measured past airway pressure and flow. Additionally or alternatively, the estimated respiratory mechanics may be used in real time for real-time estimates of muscle pressure or intrapleural pressure. As referred to herein, real time means substantially the current time or actual time during which a process or event occurs. For example, an estimate in real time is at a time within a few seconds of a measurement being received. In another example, an estimate in real time is at a time before the end of the current phase (e.g., inhalation phase or exhalation phase) of the current breath. With these concepts in mind, several examples of non-invasive estimation of intrapleural pressure methods and systems are discussed below.

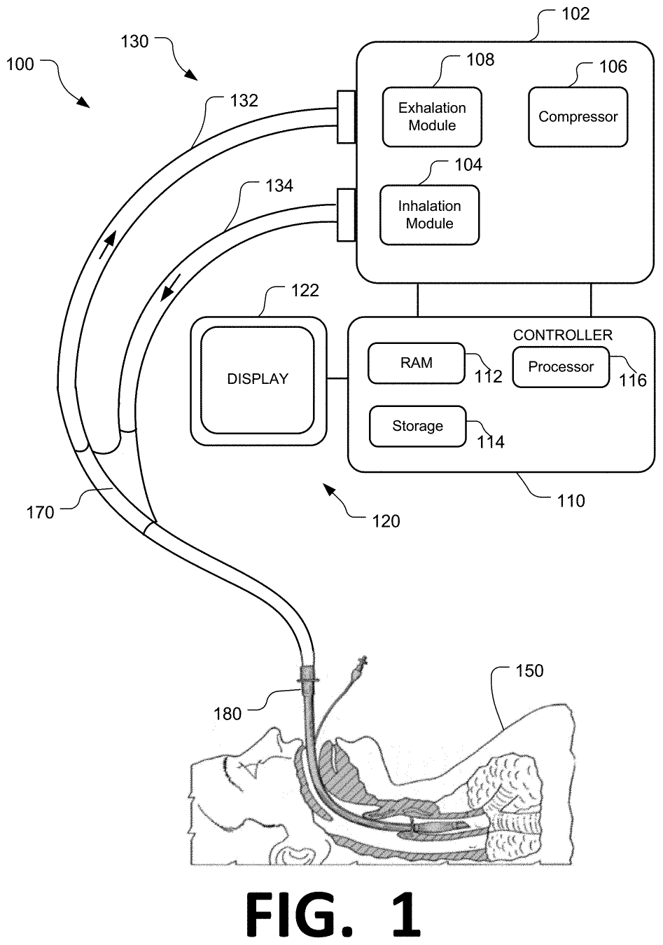

FIG. 1 is a diagram illustrating an example of a medical ventilator 100 connected to a human patient 150. A ventilator 100 may provide positive pressure ventilation to the patient 150. The ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilator system or between the ventilator system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

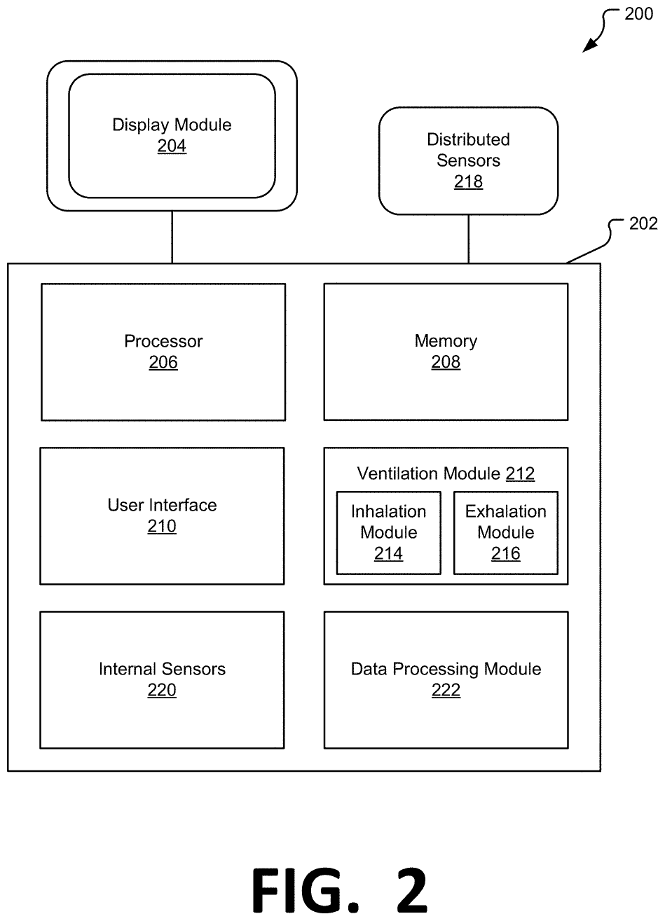

FIG. 2 is a block-diagram illustrating an example of a ventilator system 200. Ventilator system 200 includes ventilator 202 with various modules and components. That is, ventilator 202 may further include, among other things, memory 208, one or more processors 206, user interface 210, and ventilation module 212 (which may further include an inhalation module 214 and an exhalation module 216). Memory 208 is defined as described above for ventilation module 212. Similarly, the one or more processors 206 are defined as described above for one or more processors 206. Processors 206 may further be configured with a clock whereby elapsed time may be monitored by the ventilator system 200.

The ventilator system 200 may also include a display module 204 communicatively coupled to ventilator 202. Display module 204 provides various input screens, for receiving input, and various display screens, for presenting useful information. Inputs may be received from a clinician. The display module 204 is configured to communicate with user interface 210 and may include a graphical user interface (GUI). The GUI may be an interactive display, e.g., a touch-sensitive screen or otherwise, and may provide various windows (i.e., visual areas) including elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 202 may be provided, for instance by a wheel, keyboard, mouse, or other suitable interactive device. Thus, user interface 210 may accept commands and input through display module 204, such as alarm settings, drive pressure settings, past breath data (including past profiles for muscle pressure or intrapleural pressure), or other parameters or information related to the estimated intrapleural pressure. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed in the form of graphs, wave representations (e.g., a waveform), pie graphs, numbers, or other suitable forms of graphic display. For example, the data processing module 222 may be operative to determine a ventilation settings (otherwise referred to as ventilatory settings, or ventilator settings, or ventilation settings) associated with non-invasive estimation of intrapleural pressure, display information regarding the non-invasive estimation of intrapleural pressure, or may otherwise use information related to the non-invasive estimation of intrapleural pressure in connection with the ventilator, as detailed herein.

Ventilation module 212 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with non-invasive estimation of intrapleural pressure. In some cases, certain ventilation settings may be adjusted based on the exhalation flow, e.g., to adjust or improve the prescribed treatment. Ventilation settings may include inhalation flow, frequency of delivered breaths (e.g., respiratory rate, (f), tidal volume (V_{T}), PEEP level, etc.).

Ventilation module 212 may further include an inhalation module 214 configured to deliver gases to the patient and an exhalation module 216 configured to receive exhalation gases from the patient, according to ventilation settings that may be based on the exhalation flow. As described herein, inhalation module 214 may correspond to the inhalation module 104, or may be otherwise coupled to source(s) of pressurized gases (e.g., air, oxygen, and/or helium), and may deliver gases to the patient. As further described herein, exhalation module 216 may correspond to the exhalation module 108 or may be otherwise coupled to gases existing the breathing circuit.

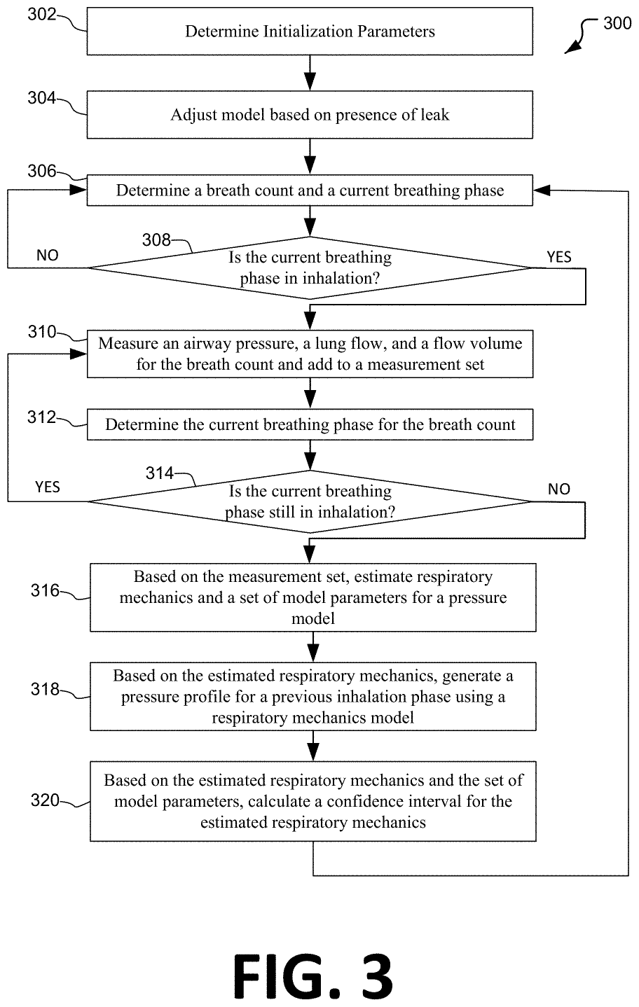

FIGS. 3-8 show example methods according to the disclosed technology. The example methods include operations that may be implemented or performed by the systems and devices disclosed herein. For example, the ventilator system depicted in FIGS. 1-2 (e.g., ventilator 100, 202) may perform the operations described in the methods. In addition, instructions for performing the operations of the methods disclosed herein may be stored in a memory of the ventilator (e.g., system memory 112, 208 described in FIGS. 1-2). FIGS. 3-8 show different aspects of the presently described technology, which are described in further detail below.

FIG. 3 is an example method 300 for non-invasively estimating intrapleural pressure (and/or muscle pressure). As further described herein, this non-invasive estimation of intrapleural pressure does not use a maneuver. Instead, a lung resistance, R, and a lung compliance, C, may be estimated based on past breath data (e.g., past airway pressure, P_{aw}, lung flow, Q, inspired lung volume, ∫Q, and PEEP), a respiratory model (e.g., the respiratory model shown in Eqn. 2, below, or the leak-compensated respiratory model shown in Eqn. 13, below), and a non-invasive muscle pressure model.

During mechanical ventilation, the total pressure applied to the respiratory system (Pₜₒₜ) with each breath is the sum of pressure developed by respiratory muscle (the negative pressure applied to the chest wall, otherwise referred to as muscle pressure or Pₘᵤₛ) and the pressure provided by the ventilator at the airway (otherwise referred to as the airway pressure, wye pressure, or P_{aw}). The muscle pressure, Pₘᵤₛ, equals the intrapleural pressure, Pₚₗ, minus the pressure gradient over the chest wall, Pow, (i.e., Pₘᵤₛ=Pₚₗ−P_{cw} and Pₚₗ=Pₘᵤₛ+P_{cw}) where P_{cw} may be determined based on the inspired lung volume, and the chest wall compliance C_{cw}. Thus, the muscle pressure, Pₘᵤₛ, and the intrapleural pressure, Pₚₗ, are related.

According to the equation of motion, the total pressure, Pₜₒₜ, is dissipated to overcome lung resistance (otherwise referred to as respiratory resistance), R, and lung compliance (otherwise referred to as respiratory compliance), C, or lung elastance (otherwise referred to as respiratory elastance or the inverse of lung compliance), E. The equation of motion is represented as follows:

Pₜₒₜₐₗ=P_{aw}+Pₘᵤₛ=R*Q+E*∫Qdt+PEEP (Eqn. 1)

where P_{aw} is the pressure measured at the patient wye, Pₘᵤₛ is the muscle pressure or pressure generated by the inspiratory muscles of the patient which is used as the proxy of the patient's muscle pressure, E is the lung elastance (the inverse of lung compliance, i.e., E=1/C), Q is the instantaneous lung flow, ∫Q is the inspired lung volume, PEEP is the positive end expiratory pressure, and R is the lung resistance.

By rearranging this equation slightly, an equation for the amount of pressure assistance at the wye (the airway pressure, P_{aw}) is then represented as follows:

P_{aw}=R*Q+E*∫Qdt+PEEP−Pₘᵤₛ (Eqn. 2)

The equation of motion (Eqn. 1) and manipulated equation of motion (Eqn. 2) are examples of equations of motion. Any suitable equation for determining a relationship between the muscle pressure (or intrapleural pressure) and airway pressure may be used. Typically, the airway pressure, P_{aw}, and the flow, Q, are measured continuously or at time intervals (e.g., every computational cycle such as a computation cycle occurring every 5 milliseconds). The ventilator estimates lung flow based on internal flow sensor measurements and estimate of circuit flow, and the ventilator estimates inspired lung volume based on the integral value of estimated lung flow. These estimates may be generated every computational cycle (e.g., 5 ms) during active ventilation. The volume, ∫Q, is computed by integrating the flow over time. As discussed above, the muscle pressure, Pₘᵤₛ, may be estimated using invasive or non-invasive means, such as placing a balloon catheter in the esophagus or using a respiratory mechanics maneuver. The present technology improves on those methods by using a muscle pressure model as further described herein.

Method 300 begins at operation 302, where initialization parameters are accessed or determined. For example, one or more of the following may occur: a default lung compliance and default lung resistance are determined, one or more pressure models (e.g., a muscle pressure model in Eqn. 5, an equation of motion in Eqn. 1 or Eqn. 12, or an airway pressure model in Eqn. 2 or Eqn. 13) is accessed or identified, a breath window size is received, and/or an error function is selected.

The default lung compliance and the default lung resistance may be determined by the ventilator based on one or more patient parameters (e.g., predicted body weight, lung condition, patient type, etc.) and/or ventilation parameters (e.g., ventilation mode, oxygen concentration, etc.). A default value for lung compliance and lung resistance may be based on a set of boundary conditions. The default value or boundary conditions may be based on a predicted body weight or ideal body weight of the patient and/or the patient type (e.g., adult, pediatric, or neonate). Alternatively, the default values or boundary conditions may be set by a clinician. In an example, the following boundary conditions may be applied to the lung elastance, E, (inverse of lung compliance) and the lung resistance, R:

3≤R≤100 cmH₂O/(L/s) (Eqn. 3)

0.01≤E=1/c≤0.2 cmH₂O/mL (Eqn. 4)

If boundary conditions are applied, then a default value of lung resistance and lung compliance may be selected from within the boundaries. For example, the default values may be at a boundary condition or in between the boundary conditions (e.g., for R, a default value of 3, 100, or a value in between 3-100).

A muscle pressure model that includes one or more coefficients is utilized in estimating intrapleural pressure values. The muscle pressure model may be any model whose coefficients are configurable to estimate a profile of the muscle pressure (or intrapleural pressure, as is related) over time during an inhalation phase of a breath of a patient. The muscle pressure model may be universal across patients or may be unique to each patient based on one or more patient parameters and/or ventilation parameters. Additionally or alternatively, the muscle pressure model may vary based on computational time or computational energy.

As an example, the muscle pressure model may be a polynomial function of any degree or any basis whose coefficients may be adjusted to accurately model the muscle pressure or intrapleural pressure during an inhalation phase of any breath. Because the muscle pressure and intrapleural pressure vary from breath to breath, the coefficients of the muscle pressure model may change from breath to breath. In an example, the muscle pressure model is a Bernstein basis polynomial of degree four, represented as follows:

Pₘᵤₛ(xₖ)=a_{n,0}*(1−xₖ)⁴+a_{n,1}*4xₖ(1−xₖ)³a_{n,2}*6xₖ²(1−xₖ)²+a_{n,3}*4xₖ³(1−xₖ)a_{n,4}*xₖ⁴eₙ(xₖ) (Eqn. 5)

where there are N samples of airway pressure measured during the inhalation phase of the n^{th} breath that spans a total sample time interval Tₜₒₜ, and xₖ is time normalized over the time interval. The coefficients of the fourth order Bernstein basis muscle pressure model may be unique from breath to breath and are represented in the above example as a_{n,0}, a_{n,1}, a_{n,2}, a_{n,3}, and a_{n,4}. The muscle pressure model error is represented as eₙ(xₖ) and may also vary from breath to breath.

The normalized time, xₖ, is the time at which a sample is measured, divided by the total time interval Tₜₒₜ. The N samples measured over the time interval may be associated with a time vector, \vec{t} The normalized time is represented as follows:

$${x}_{k} = \frac{\vec{t}}{{T}_{t o t}} \qquad ( Eqn . 6 )$$

In an example, the N samples may be measured continuously or at evenly spaced time increments, Tₛ (e.g., 5 ms). If measurements are taken at evenly spaced time intervals, Tₛ, then the sample time sequence for an inhalation phase of a breath is \vec{t}=[0, Tₛ, 2Tₛ, . . . , (N−1)Tₛ] (where Tₜₒₜ=(N−1)Tₛ). The normalized time, xₖ, may then be simplified as follows:

$${x}_{k} = \frac{\vec{t}}{{T}_{t o t}} = \frac{[ 0 , {T}_{s} , 2 {T}_{s} , . . . , ( N - 1 ) {T}_{s} ]}{( N - 1 ) {T}_{s}} = [ 0 , \frac{1}{N - 1} , \frac{2}{N - 1} , . . . , 1 ] \qquad ( Eqn . 7 )$$

Although the example muscle pressure model represented in Eqn. 5 is a Bernstein basis polynomial of the fourth degree, the muscle pressure model may be a linear model (or a series of piece-wise linear models) or any other degree of polynomial with any basis.

As further described herein, the muscle pressure model has one or more adjustable model parameters (e.g., coefficients, such as a_{n,0}, a_{n,1}, a_{n,2}, a_{n,3}, and a_{n,4} in Eqn. 5, and/or error parameters, such as eₙ(xₖ) in Eqn. 5) that may be adjusted to uniquely model the intrapleural pressure for each breath. One or more of the model parameters may be associated with one or more parameter constraints. A parameter constraint may be a range of values, a set of values, a minimum value, a maximum value, a threshold value, a sign (e.g., positive or negative), a sign of a first derivative, a sign of a second derivative, or any other constraint on a model parameter. The parameter constraint(s) may be based on a specific sample time, t, or a specific normalized time, xₖ. The parameter constraint(s) may be predetermined or specified. Alternatively, the parameter constraint(s) may be based on past data collected or measured.

In an example where the muscle pressure model is the fourth-degree Bernstein basis polynomials described in Eqn. 5, the muscle pressure model includes the following model parameters: a_{n,0}, a_{n,1}, a_{n,2}, a_{n,3}, a_{n,4}, eₙ. For instance, the following parameter constraints may be applied:

$$\begin{matrix}at {x}_{k} = 0 , {P}_{mus} ( 0 ) = {a}_{0} ∈ [ - 5 , 10 ] & ( Eqn . 8 A ) \\ at {x}_{k} = 1 , {P}_{mus} ( 1 ) = {a}_{4} ∈ [ - 5 , 50 ] & ( Eqn . 8 B ) \\ at {x}_{k} = 0 , \frac{d {P}_{mus}}{d {x}_{k}} = - 4 {a}_{0} + 4 {a}_{1} \geq 0 & ( Eqn . 8 C ) \\ at {x}_{k} = 0 , \frac{{d}^{2} {P}_{mus}}{d {x}_{k}^{2}} = 12 {a}_{0} - 24 {a}_{1} + 12 {a}_{2} \leq 0 & ( Eqn . 8 D ) \\ at {x}_{k} = 1 , \frac{{d}^{2} {P}_{mus}}{d {x}_{k}^{2}} = 12 {a}_{2} - 24 {a}_{3} + 12 {a}_{4} \leq 0 & ( Eqn . 8 E )\end{matrix}$$

where xₖ=0 represents the beginning of the inhalation phase for a breath and xₖ=1 represents the end of the inhalation phase for the breath.

As further described herein, adjustable model parameters of the muscle pressure model may vary from breath to breath based on measured ventilation data (e.g., airway pressure, P_{aw}, lung flow, Q, inspired lung volume, ∫Q). Although adjustable model parameters of the muscle pressure model may change from breath to breath, the lung resistance and lung compliance may be substantially constant over a number of prior breaths. The number of prior breaths to estimate a common lung resistance and a common lung compliance (otherwise referred to herein as a breath window) may be predetermined or set by a clinician. In an example, the breath window may be between two to ten breaths.

The adjustable model parameters of the muscle pressure model for each breath of the breath window, along with a lung resistance and lung compliance common among each breath of the breath window, may be estimated using an error function. The error function may be represented as follows:

S(θ)=Σₖ₌₀^{N-1}½|\hat{P}_{aw}(xₖ)−P_{aw}(xₖ)|² (Eqn. 9)

where, θ is the parameter vector that includes the adjustable model parameters for each breath of the breath window, the lung resistance, and the lung compliance or lung elastance; \hat{P}_{aw}(xₖ) is the estimated airway pressure at normalized time xₖ based on the parameter vector, θ, the muscle pressure model associated with the adjustable model parameters, and the respiratory model of Eqn. 2 (or the leak-compensated respiratory model shown in Eqn. 13); and P_{aw}(xₖ) is the airway pressure measured at normalized time xₖ. For example, if the breath window is three breaths (including breaths n=1, n=2, and n=3) and the muscle pressure model is a Bernstein basis polynomial described in Eqn. 5, then the parameter vector after the third breath, n=3, may be represented as follows:

θ=[R E a₁₀a₁₁a_{1,2}a_{1,3}a₁₄a_{2,0}a_{2,1}a_{2,2}a_{2,3}a₂a_{3,0}a_{3,1}a_{3,2}a_{3,3}a_{3,4}] (Eqn. 10)

When initiating ventilation of a patient, the number of prior breaths available may be less than the breath window. If the quantity of breaths for which ventilation data has been measured is less than the breath window, then breath window may be an initiation breath window equal to the quantity of available prior breaths. For example, if the breath window is five breaths and ventilation data has been measured for three breaths, then the initiation breath window is three breaths. After a fourth breath, the initiation breath window is four breaths. After a fifth breath, the breath window is five breaths because the quantity of available prior breaths is greater than or equal to the breath window and an initiation breath window is no longer implemented. The parameter vector, θ, may be adjusted until the error function (e.g., the error function of Eqn. 9) is minimized. Any error optimization method may be applied to minimize the error of the error function, such as the Levenberg-Marquart method.

Other initialization parameters, functions, or values may also be set or determined at operation 302. For example, default adjustable model parameters may be determined based on the parameter constraints (e.g., the parameter constraints in Eqns. 8A-E), a measurement interval to measure ventilation parameters (e.g., 5 ms) may be set, or storage arrays may be constructed for permanently or temporarily storing measured ventilation data, adjustable model parameters, or values or parameters associated with estimating intrapleural pressure.

At operation 304, a model is adjusted based on the presence of a leak. In the presence of a leak, the lung compliance and lung resistance estimation may be compensated. In order to compensate for leakage in the circuit, an ongoing calculation of leakage may be taken into account. An example of methods and systems for compensating for leaks during PAV are discussed in U.S. Pat. No. 8,978,650, filed Apr. 26, 2013, titled Leak-Compensated Proportional Assist Ventilation, which is hereby incorporated by reference in its entirety.

The lung compliance and lung resistance estimation may be compensated by adjusting the lung flow, Q, and inspired lung volume, ∫Q to compensate for a leak flow, Qₗₑₐₖ. The leak flow, Qₗₑₐₖ, is estimated by ventilator-specific leak compensation software or algorithms. An example of methods and systems determining leak flow during ventilation are discussed in U.S. Pat. No. 9,498,589, filed Dec. 31, 2011, titled Methods and Systems for Adaptive Base Flow and Leak Compensation, which is hereby incorporated by reference in its entirety.

Alternatively, leak may be incorporated into the equation of motion (e.g., Eqn. 1) based on the following relationship between the leak flow, Qₗₑₐₖ, and airway pressure, P_{aw}, as a model of leak through an orifice:

Qₗₑₐₖ=k₁×(P_{aw})^{1/2}k₂×(P_{aw})^{3/2} (Eqn. 11)

where k₁ and k₂ are constants based on the breathing circuit and/or ventilator system. The constants, k₁ and k₂, may be estimated based data. The leak flow, Qₗₑₐₖ, is pressure driven such that as airway pressure increases, the leak flow increases.

The equation of motion of Eqn. 1 may be modified by subtracting the leak flow from the lung flow, where the leak flow is based on the above relationship in Eqn. 11. The leak-compensated equation of motion may be represented as follows:

$${P}_{t o t} = {P}_{a w} + {P}_{m u s} = R * [ Q - {k}_{1} * {( {P}_{a w} )}^{\frac{1}{2}} - {k}_{2} * {( {P}_{a w} )}^{\frac{3}{2}} ] + E * \int [ Q - {k}_{1} * {( {P}_{a w} )}^{\frac{1}{2}} - {k}_{2} * {( {P}_{a w} )}^{\frac{3}{2}} ] d t + P E E P \qquad ( Eqn . 12 )$$

By manipulating this equation, the leak-compensated airway pressure may then be represented as follows:

$${P}_{a w} = R * [ Q - {k}_{1} * {( {P}_{a w} )}^{\frac{1}{2}} - {k}_{2} * {( {P}_{a w} )}^{\frac{3}{2}} ] + E * \int [ Q - {k}_{1} * {( {P}_{a w} )}^{\frac{1}{2}} - {k}_{2} * {( {P}_{a w} )}^{\frac{3}{2}} ] d t + P E E P - {P}_{m u s} \qquad ( Eqn . 13 )$$

If no leak is present, then the relationships described for the equation of motion of Eqn. 1 and the airway pressure model of Eqn. 2 are used. Alternatively, if a leak is present or a leak-based option is selected on the ventilator, then the lung flow is adjusted by the leak flow in the relationship of Eqn. 12 and Eqn. 13. For example, if values for the leak flow over time are estimated, then the lung flow values may be adjusted by the estimated leak flow values. In another example, if the leak flow is related to the airway pressure using the relationship in Eqn. 11, then the lung flow may be adjusted based on that relationship, such as the adjustments shown to the leak-compensated equation of motion in Eqn. 12 and the leak-compensated airway pressure model in Eqn. 13.

At operation 306, a breath count, n, and a current breathing phase (i.e., inhalation or exhalation) are determined. The breath count, n, may be associated with ventilation data. A breath begins at the start of the inhalation phase and continues until the start of the next inhalation phase. For example, a first breath, n=1, starts at the beginning of the first inhalation phase and continues through the first exhalation phase. At the start of the second inhalation phase, the first breath ends and the second breath, n=2, begins and continues through the second exhalation phase ending at the start of the third inhalation phase. The current breathing phase may be automatically determined by the ventilator based on measured ventilation parameters.

At determination 308, the current breathing phase is evaluated. The ventilator may determine if the current breathing phase is inhalation. If the current breathing phase is exhalation, flow branches “NO” back to operation 306 where the breath count and current breathing phase are determined. Operations 306 and 308 may repeat until the current breathing phase is determined to be inhalation.

If the current breathing phase is inhalation, then flow branches “YES” to operation 310 where an airway pressure, P_{aw}, a lung flow, Q, and a flow volume, ∫Q, are measured for the breath count, n. The measurements at each of the N samples may be added to a measurement set. Each breath may have a unique measurement set (e.g., N samples all from a single breath n). Alternatively, the measurement set may include measurements from a plurality of breaths (e.g., a subset of the N samples is from breath n=1, another subset of the N samples is from breath n=2, etc.). The measurement set of N samples may associate each measurement with a measurement time and a breath count, n, at which the measurement was received. The measurement time may span from the start of the inhalation phase of the breath count until the measurement is recorded. For example, the measurement time associated with a measurement is the time elapsed from the beginning of the inhalation phase. Thus, if a measurement for a sample is taken at the start of the inhalation phase for the second breath, the associated measurement time is t=0 and the associated breath count is n=2. Alternatively, the measurement time may span from a single reference point and may be adjusted based on the time at which the inhalation phase begins for the breath associated with a measurement.

At operation 312, the current breathing phase for the breath count, n, is determined. As further described herein, the breathing phase may be inhalation or exhalation. A ventilator may determine the current breathing phase based on ventilation data.

At determination 314, the current breathing phase is evaluated. For the breath count, n, a ventilator may determine if the current breathing phase is still inhalation, or if the breath has progressed to the exhalation phase. If the current breathing phase is still inhalation (i.e., the patient is still inhaling for that breath), then flow progresses “YES” back to operation 310. Operations 310-314 may repeat until the end of the inhalation phase for that breath. Thus, the ventilator may continue to measure ventilation parameters during the inhalation phase until the breath progresses to the exhalation phase (e.g., receiving N samples of ventilation data during the inhalation phase of the breath n).

If the current breathing phase is not inhalation (i.e., the inhalation phase for that breath has ended or entered the exhalation phase), then flow proceeds to operation 316 where a lung resistance, a lung compliance, and a set of model parameters for a muscle pressure model for the breath are estimated, based on the measurement set. The muscle pressure model may be any model used to estimate the intrapleural pressure of the patient over the inhalation phase of a breath, n, as further described herein. If the measurement set includes measured ventilation data for two or more breaths (e.g., N samples from breath n=1 and M samples from breath n=2), then two or more sets of model parameters for the muscle pressure model may be estimated, with each set of model parameters associated with each breath (e.g., a first set of model parameters for breath n=1 and a second set of model parameters for breath n=2). As further described herein, model parameters may be coefficients, error functions, or other adjustable values aspects of the muscle pressure model estimating the intrapleural pressure. The lung resistance and the lung compliance may be assumed to be constant over the breath window that may be set at operation 302.

The measurement set with N samples for the breath n may be augmented by normalizing the measurement set by time. As described herein, the measurement time associated with each measurement may be based on, or adjusted by, the start of the inhalation phase for the associated breath. The total time for the inhalation phase of the associated breath may be used to augment the associated time for each measurement. For example, the measurement time may be normalized over the inhalation phase based on the relationship described in Eqn. 6 or Eqn. 7.

The lung resistance, lung compliance or lung elastance, and one or more sets of model parameters for the muscle pressure model may be estimated based on the error function set at operation 302. For example, a parameter vector, θ, including the lung resistance, lung compliance or lung elastance, and one or more sets of model parameters, may be adjusted according to the Levenberg-Marquart method (or any other error reduction method) until the error function is minimized (e.g., for Eqn. 9, until the estimated airway pressure using the muscle pressure model and estimated lung resistance and lung compliance is approximately equal to the measured airway pressure at each normalized time for each breath). The values of the parameter vector may be limited based on parameter constraints (e.g., parameter constraints defined in Eqns. 8A-E). In an example, initial parameter values may be initial values for estimating the parameter vector, θ, using an error reduction method. The initial parameter values may be based on parameter constraints (e.g., upper boundary, lower boundary, mean, etc.) or may be based on prior estimations from one or more prior breaths of the patient.

At operation 318, based on the estimated lung resistance and the estimated lung compliance, a profile of muscle pressure is generated for the breath count, n, based on a respiratory mechanics model. The equation of motion shown in Eqn. 1 (or the leak-compensated equation of motion in Eqn. 12) may be manipulated to represent the muscle pressure:

Pₘᵤₛ(t)=R*Q(t)+E*∫Q(t)dt+PEEP−P_{aw}(t) (Eqn. 14a)

A profile of intrapleural pressure may be generated for the breath count, n, based on pressure gradient over the chest wall, P_{cw}:

$${P}_{p l} ( t ) = {P}_{m u s} ( t ) + {P}_{c w} ( t ) = {P}_{m u s} ( t ) + ( \frac{1}{{C}_{c w}} ) * \int Q ( t ) d t \qquad ( Eqn . 14 b )$$

As described above, the lung flow, Q, inspired lung volume, ∫Q, PEEP, and airway pressure, P_{aw}, are each known or measured at measurement time, t. With the estimated lung resistance, R, and estimated lung elastance, E, determined at operation 314, the muscle pressure, Pₘᵤₛ, may be modeled over time, t, using Eqn. 14a. Additionally, using Eqn. 14b, the intrapleural pressure, Pₚₗ, may be modeled over time, t, based on the muscle pressure, Pₘᵤₛ, and the chest wall pressure, P_{cw}, (which is estimated by dividing the inspired lung volume by the chest wall compliance, C_{cw}). Thus, the muscle pressure Pₘᵤₛ and/or the intrapleural pressure, Pₚₗ, may be modeled for prior breaths based on the measured ventilation data, or in real time.

At operation 320, a confidence interval is calculated for the lung resistance and the lung compliance. The confidence interval may be calculated based on the estimated lung resistance, the estimated lung compliance, and the estimated set of model parameters. The error function (e.g., the error function shown in Eqn. 9) is associated with a curvature matrix including the second derivatives of the error function with respect to the parameter vector, θ. The curvature matrix is approximated with the first derivatives, represented as follows:

$$α = [ \frac{1}{2} \frac{{\partial }^{2} S ( θ )}{\partial {θ}_{k} \partial {θ}_{l}} ] \approx [ \frac{\partial S ( θ )}{\partial {θ}_{k}} \cdot \frac{\partial S ( θ )}{\partial {θ}_{l}} ] \qquad ( Eqn . 15 )$$

A covariance matrix, CV, of the model parameters estimated may be useful for estimating a confidence interval. This matrix is the inverse of the curvature matrix, represented as follows:

CV=α⁻¹ (Eqn. 16)

The error function can be represented by the contours of a surface. If the error function is represented in linear parameter vectors, θ, the surface contours are be ellipsoidal and have a single global minimum, S(θₘᵢₙ), at the location defined by the least square estimator θₘᵢₙ. If the model is nonlinear, the contours are not ellipsoidal but tend to be irregular with one or more local minimal points. A confidence contour is defined by setting S(θ) equal to a constant. This may not be feasible in a multi-dimensional nonlinear case. Under the assumption that the linearized form of the model is valid around θₘᵢₙ, which is the estimated parameter vector, θ, the ellipsoidal confidence region is obtained by the following relationships:

$$\begin{matrix}{( θ - {θ}_{min} )}^{T} \cdot {( α )}_{θ = {θ}_{min}} \cdot ( θ - {θ}_{min} ) \leq p \cdot {s}^{2} \cdot F ( p , m - p , 1 - α ) & ( Eqn . 18 ) \\ {s}^{2} = \frac{S ( {θ}_{min} )}{( m - p )} & ( Eqn . 18 )\end{matrix}$$

where, p is the total number of parameters in the parameter vector, θ; m is the total number of measurements; 100×(1−α)% is the confidence level (e.g., α=0.05 represents 95% confidence); and F(p, m−p, 1−α) is the F inverse cumulative distribution function.

Operations 306-320 may repeat from breath to breath. The breath count, n, is updated at the initiation of a new inhalation phase (i.e., determining a transition from an exhalation phase of breath n to an inhalation phase of breath n+1). For the new breath, a measurement set is collected (e.g., M samples over the inhalation phase of breath n+1). The parameter vector may be estimated based on the current measurement set for breath n+1 and one or more past measurement sets for breath n or preceding breaths, such that measured ventilation data from two or more breaths (e.g., M samples from breath n+1, N samples from breath n, etc.) may be used to re-estimated the lung resistance, lung compliance, and two or more sets of model parameters for the muscle pressure model for the two or more breaths (e.g., breath n and breath n+1).

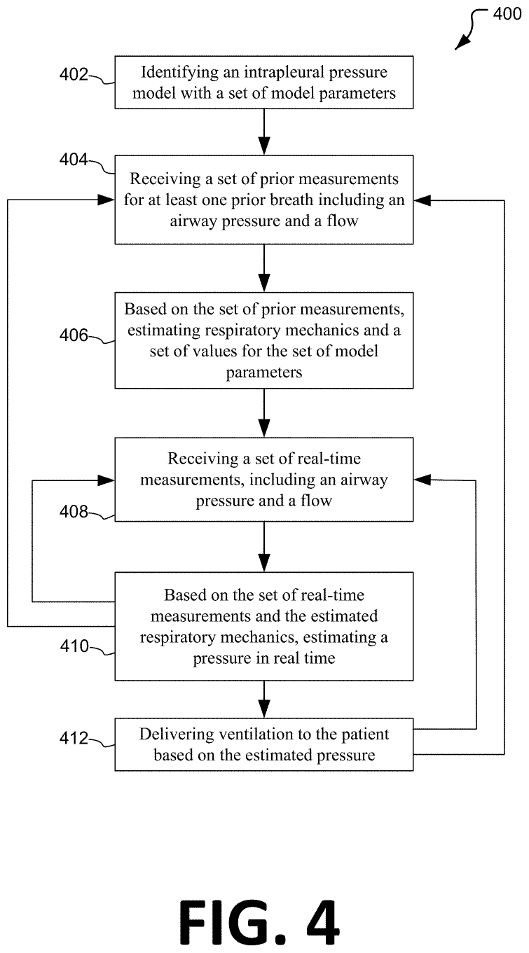

FIG. 4 is an example method 400 for real-time, non-invasive estimation of intrapleural pressure (otherwise referred to as a proxy for intrapleural pressure, or correlated intrapleural pressure). The method 400 may utilize estimations of patient respiratory mechanics from past breath ventilation data to estimate intrapleural pressure in real time. Method 400 may implement one or more aspects of method 300 shown in FIG. 3 for estimating intrapleural pressure or muscle pressure. Method 400 further includes operations relating to real-time estimation of intrapleural pressure or muscle pressure.

At operation 402, a muscle pressure model is identified, the muscle pressure model having a set of model parameters (e.g., coefficients, error functions, or parameter adjustable to uniquely model an inhalation phase of a patient from breath to breath). The muscle pressure model may have the same or similar features of the muscle pressure models described herein. For example, the muscle pressure model may be a polynomial of any basis or any degree, such as a Bernstein basis polynomial of degree four.

At operation 404, a set of prior measurements for prior breath(s) is received, the set of prior measurements including airway pressure measurement(s) and flow measurement(s). The flow measurement(s) is used to calculate inspired lung volume by integrating the flow over time. The set of prior measurements are associated with one or more breaths. For example, a first subset of the set of prior measurements may be associated with a first breath and a second subset of the set of prior measurements may be associated with a second breath. Each measurements of the set of prior measurements is measured during an inhalation phase of a breath.

At operation 406, based on the set of prior measurements, a lung compliance, a lung resistance, and a set of values for the set of model parameters is estimated. The estimation may be based on an error reduction or error minimization procedure, such as the estimation performed at operation 314 of FIG. 3. If the set of prior measurements includes ventilation data from two or more breaths, then model parameters for each of the two or more breaths may be estimated. For example, if the set of prior measurements includes a first subset from a first breath and a second subset from a second breath, then the set of values for the set of model parameters may include a first parameter set associated with the first breath and a second parameter set associated with the second breath. The lung compliance and the lung resistance are assumed to be common among ventilation data included in the set of prior measurements. Thus, a single value for the lung compliance and a single value for the lung resistance may be estimated, even when ventilation data from two or more breaths is included in the set of prior measurements.

At operation 408, a set of real-time measurements is received during a phase of a breath (e.g., inhalation phase or exhalation phase). The set of real-time measurements include airway pressure measurement(s) and flow measurement(s). The real-time flow measurement(s) may be integrated over time to determine a real-time inspired lung volume. Unlike the set of prior measurements, which are measurements taken during one or more prior breaths, the set of real-time measurements are taken substantially in real time during a current or ongoing breath.

At operation 410, a real-time muscle pressure (or real-time intrapleural pressure, as is related to the muscle pressure) is estimated as a proxy for intrapleural pressure, based on the set of real-time measurements, the estimated lung compliance, and the estimated lung resistance. The muscle pressure is estimated during the same phase of the breath of which the set of real-time measurements is received at operation 408 (e.g., substantially in real time). A predetermined relationship between the intrapleural pressure, the set of real-time measurements, the lung compliance, and the lung resistance may be known. For example, an equation of motion (e.g., the equation of motion in Eqn. 1 or leak-compensated equation of motion Eqn. 12) may be used to determine a relationship between the intrapleural pressure and the known ventilation data. By substituting the set of real-time measurements, the estimated lung compliance, and the estimated lung resistance into the relationship, a proxy for intrapleural pressure, or correlated intrapleural pressure, may be determined in real time.

Operations 408-410 may repeat as measured ventilation data changes in real time. For example, as the inhalation phase of a breath progresses, the measured airway pressure and measured flow varies. As the airway pressure and/or flow varies, the real time muscle pressure or intrapleural pressure is updated accordingly. As further described herein, the real time estimation of intrapleural pressure may be implemented for other purposes, such as monitoring and/or alarming off of patient drive pressure, transpulmonary pressure, and intrapleural pressure, or for trigger detection during inhalation or exhalation.

Further, the non-invasive estimation of intrapleural pressure allows for triggering. If the patient's lung resistance during exhalation is substantially similar to the lung resistance during inhalation, then aspects described herein may be used to dynamically estimate the intrapleural pressure during the exhalation phase. For example, breath triggering may be based on the value of intrapleural pressure or the sign of the derivative of intrapleural pressure, or a combination. The non-invasive estimation of intrapleural pressure during the exhalation phase may be used for trigger detection that traditionally requires invasive estimations, such as neutrally adjusted ventilatory assist (NAVA).

Additionally or alternatively, the muscle pressure or intrapleural pressure for past breaths may be modeled based on the set of values for the set of model parameters and the muscle pressure model. For example, assuming that the set of prior measurements includes ventilation data for a first breath and a set of values associated with the first breath are estimated for the set of model parameters, the model parameters may be included in the muscle pressure model to estimate an intrapleural pressure at any time during the inhalation phase of the first breath. Alternatively, the muscle pressure or intrapleural pressure for past breaths may be modeled based on the set of prior measurements and the estimated lung resistance and estimated lung compliance. For example, the muscle pressure may be determined at any given time based on measurements of the set of prior measurements taken at that time and the estimated respiratory mechanics, using the relationship defined in an equation of motion (e.g., Eqn. 1 or Eqn. 12).

Operations 404-410 may repeat as required or desired. For example, the set of prior measurements may be updated from breath to breath. As additional ventilation data is measured, the lung compliance, the lung resistance, and the set of values for the set of model parameters is re-estimated. Aspects of the intrapleural pressure may be monitored over a series of breaths, over several hours, over multiple days, or any other period of time. Monitoring of the intrapleural pressure over time may assist in clinical assessment of a patient for purposes such as weaning off of ventilation or extubation readiness. Parameter trending may be used in the clinical assessment, including maximum intrapleural pressure from breath-to-breath, variation in one or more values of intrapleural pressure across breaths, change in profile or shape from breath-to-breath, etc.

Other parameters relating to the non-invasive estimation of intrapleural pressure may also be monitored over time for purposes such as parameter determination or parameter trending. For example, other parameters include drive pressure or maximum transpulmonary pressure, lung resistance, lung compliance, lung elastance, and work of breathing (“WOB”), etc.

The drive pressure, or driving pressure, P_{drive}, is used as a substitute for the strain applied to the lungs of a patient due to lung expansion. The drive pressure is sometimes defined as the combined plateau pressure (at the wye) and estimated total PEEP pressure at the end of exhalation (which is an estimated lung pressure or alveolar pressure). Drive pressure is based on a static end volume estimate multiplied by lung elastance, E (or the ratio between tidal volume and lung compliance, C). Therapeutically, the drive pressure is ideally the maximum transpulmonary pressure over time. With the present technology, transpulmonary pressure, P_{L} (i.e., the difference between the airway pressure and the intrapleural pressure, or P_{L}=P_{aw}−Pₚₗ), may be estimated non-invasively using the measured airway pressure and the non-invasive estimation of intrapleural pressure. The maximum estimated transpulmonary pressure, P_{L}, and/or drive pressure, P_{drive}, may be set as an alarm setting to promote lung-protective and diaphragm-protective ventilation. For example, an alarm may sound when the estimated transpulmonary pressure exceeds the drive pressure (i.e., P_{L}=P_{aw}−Pₚₗ=P_{aw}−Pₘᵤₛ−P_{cw}>P_{drive}). Alarming based on transpulmonary pressure and/or drive pressure may be preferable to alarming based on airway pressure, as higher airway pressures may be desirable for patients with larger muscle pressure (i.e., more negative values of intrapleural pressure). Alarm settings based on limiting the transpulmonary pressure may prevent patient self-inflected lung injury (P-SILI) while allowing higher airway pressures when desired.

At operation 412, ventilation is delivered to the patient on the real-time muscle pressure (or proxy for the muscle pressure) or real-time intrapleural pressure (or proxy for the intrapleural pressure). For example, inhalation flow or exhalation pressure may be adjusted based on the estimated intrapleural pressure. A change in the muscle pressure or change may be associated with a proportional adjustment of the amount of pressure support provided by ventilation (e.g., a support setting in PAV, a change in the inhalation flow and/or exhalation pressure). A change intrapleural pressure may be associated with a change in transpulmonary pressure or drive pressure, as further described herein, and may be associated with an adjustment of the amount of pressure support provided. Additionally or alternatively, ventilation settings may be based on estimated lung resistance, estimated lung compliance, WOB of the patient, WOB of the ventilator, patient drive pressure, transpulmonary pressure, trigger detection, etc. Additionally or alternatively, the ventilator may update alarm settings based on the real time proxy for intrapleural pressure, or estimations of past intrapleural pressure.

Operations 404-412 or operations 408-412 may repeat as required or desired. For example, ventilation delivered to the patient may be adjusted based on changes in the set of real-time measurements, the real-time muscle pressure or the real-time intrapleural pressure, the estimated lung compliance, the estimated lung resistance, the estimated set of values for the set of model parameters, and the set of prior measurements.

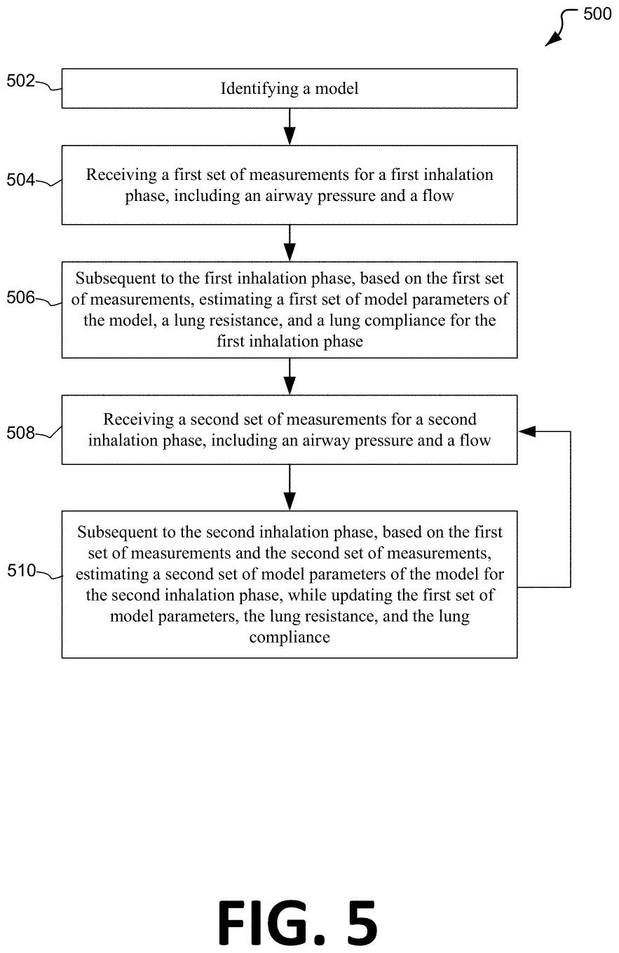

FIG. 5 is an example method 500 for non-invasive estimation of intrapleural pressure from breath to breath, based on measurements received from a plurality of inhalation phases. Method 500 shows how the estimated respiratory mechanics and estimated model parameters are updated when additional ventilation data (e.g., airway pressure(s) and flow(s)) associated with an additional breath, is received. At operation 502, a muscle pressure model is identified. This operation 502 may be the same or similar to identifying a muscle pressure model in operation 402 of FIG. 4.

At operation 504, a first set of measurements for a first inhalation phase of the patient is received, the first set of measurements including airway pressure measurement(s) and flow measurement(s). The first set of measurements may also include inspired lung volume(s) that may be determined based on the flow measurement(s). The first set of measurements may be associated with the first inhalation phase, or a first breath that includes the first inhalation phase. Each measurement of the first set of measurements may be associated with a time during the first inhalation phase.

At operation 506, subsequent to the first inhalation phase and based on the first set of measurements, a first set of model parameters of the muscle pressure model, a lung resistance, and a lung compliance for the first inhalation phase are estimated. This may be the same or similar to operation 406 of FIG. 4, such that the first set of measurements is the set of prior measurements. The first set of model parameters, lung resistance, and lung compliance may be included in a parameter vector. The parameter vector may be estimated using methods described herein.

At operation 508, a second set of measurements for a second inhalation phase of the patient is received, the second set of measurements including airway pressure measurement(s) and flow measurement(s). The second inhalation phase is a portion of a second breath occurring after the first breath. The second set of measurements may also include inspired lung volume(s) that may be determined based on the flow measurement(s). The second set of measurements may be associated with the second inhalation phase, or the second breath that includes the second inhalation phase. Each measurement of the second set of measurements may be associated with a time at which the measurement was taken during the second inhalation phase.

At operation 510, subsequent to the second inhalation phase and based on the first set of measurements and the second set of measurements, a second set of model parameters of the muscle pressure model for the second inhalation phase is estimated, while updating the first set of model parameters, the lung resistance, and the lung compliance. The lung resistance and the lung compliance are common to the first inhalation phase and the second inhalation phase. The parameter vector may be updated to include the second set of model parameters, in addition to the previously estimated first set of model parameters, lung resistance, and lung compliance. Based on the first set of measurements and the second set of measurements, the updated parameter vector may be re-estimated. Thus, estimated values for the parameter vector may change as new measurements are received. Depending on the method of estimation, the previously estimated values may be used as an initial guess for re-estimation. For example, the estimated values for the first set of model parameters, lung resistance, and lung compliance estimated at operation 506 may be used as initial guesses during re-estimation at operation 510.

One or more of the estimated second set of model parameters and updated first set of model parameters, lung resistance, and lung compliance may be used by the ventilator or a clinician. For example, based on the muscle pressure model and the second set of model parameters, a value of intrapleural pressure for the second inhalation phase may be generated at any point in time during the second inhalation phase. Additionally or alternatively, the updated lung resistance and updated lung compliance may be used to estimate a muscle pressure or intrapleural pressure in real time, such as described in operations 408-410 in FIG. 4.

Operations 508-510 may repeat as required or desired. For example, a third set of measurements may be received for a third inhalation phase. A third set of model parameters for the third inhalation phase may be estimated while updating the first set of model parameters, the second set of model parameters, the lung resistance, and the lung compliance (such that the lung resistance and lung compliance are common to the first inhalation phase, the second inhalation phase, and the third inhalation phase). This may continue for a fourth breath, fifth breath, etc.

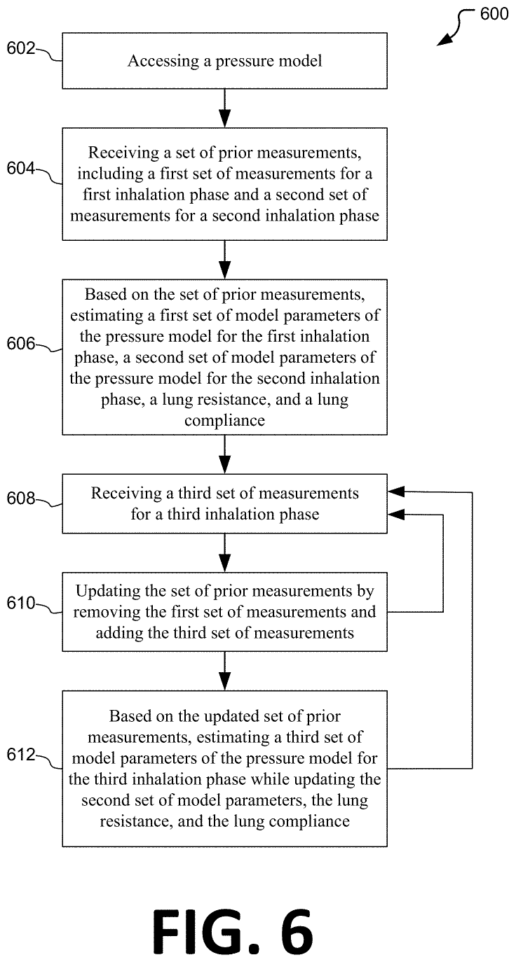

FIG. 6 is an example method for non-invasive estimation of intrapleural pressure from breath to breath based on measurements received from a subset of prior inhalation phases. While method 500 of FIG. 5 shows additional ventilation data from new breaths being received and used to estimate model parameters, lung compliance, and lung resistance, method 600 shows a method for estimating based on a subset of that past ventilation data. Thus, although ventilation data for a plurality of past breaths is known, some of that ventilation data may not be used to estimate model parameters, lung compliance, and lung resistance.

At operation 602, a muscle pressure model is accessed. As described herein, the muscle pressure model may be any polynomial of any basis or degree. The muscle pressure model may be accessed by the ventilator (e.g., ventilator 100, 202) automatically, or as specified by a clinician. For example, the ventilator may automatically access the muscle pressure model when operating in specific modes of ventilation or when a clinician requests a current or past intrapleural pressure of the patient or other parameters associated with the intrapleural pressure.

At operation 604, a set of prior measurements is received, including a first set of measurements for a first inhalation phase and a second set of measurements for a second inhalation phase a patient. Each measurement of the set of prior measurements may be associated with a time at which the measurement was taken, and the inhalation phase (or breath count) during which the measurement was taken. Although two inhalation phases are used in this example, the set of prior measurements may include measurements or measurement sets from any amount of prior breaths.

At operation 606, a first set of model parameters for the first inhalation phase, a second set of model parameters for the second inhalation phase, a lung resistance, and a lung compliance (together, the adjustable parameters, otherwise referred to as the parameter vector) are estimated, based on the set of prior measurements. As further described herein, the model parameters are associated with the muscle pressure model such that the muscle pressure model is adjustable to uniquely model the first inhalation phase and the second inhalation phase. The adjustable parameters may be estimated based on estimation methods described herein (e.g., operations 314, 406, 506).

At operation 608, a third set of measurements for a third inhalation phase of the patient are received. The third inhalation phase occurs subsequent to the first inhalation phase and the second inhalation phase. Similar to the first set of measurements and the second set of measurements, each measurement of the third set of measurements may be associated with a time at which the measurement was taken, and the inhalation phase (or breath count) during which the measurement was taken (e.g., for the third set of measurements, the third inhalation phase or the third breath).

At operation 610, the set of prior measurements is updated by removing the first set of measurements and adding the third set of measurements. The set of prior measurements may include a subset of prior breath ventilation data. In an example, when the newest set of measurements is received (e.g., the third set of measurements), the oldest set of measurements may be removed from the set of prior measurements (e.g., the first set of measurements). Thus, in this example, the set of prior measurements includes measured ventilation data from the two most recent breaths at any given time. Although, in this example, measured ventilation data from two breaths is included in the set of prior measurements (e.g., the first and second, or second and third), any number of breaths may be included. The set of prior measurements may include ventilation data from breaths that are non-consecutive (e.g., the first breath and the third breath, every fourth breath, every seventh breath, etc.). Alternatively, the set of prior measurements may be updated after each breath to include ventilation data from a specified amount of prior, consecutive breaths (e.g., after the third breath, the set of prior measurements includes ventilation data measured during the second breath and the third breath).

At operation 612, based on the updated set of prior measurements, a third set of model parameters of the muscle pressure model for the third inhalation phase is estimated, while updating the second set of model parameters, the lung resistance, and the lung compliance. As further described herein, the model parameters are associated with the muscle pressure model such that the muscle pressure model is adjustable to uniquely model different inhalation phases for which ventilation data is measured. Thus, as the set of prior measurements is updated, the model parameters also change. In this case, because the updated set of prior measurements includes the third set of measurements and the second set of measurements, a third set of model parameters and a second set of model parameters, respectively, estimated to model the third inhalation phase and the second inhalation phase. Because the estimation is based on a different, updated set of prior measurements, the second set of model parameters, the lung resistance, and the lung compliance may be different from prior estimations. This operation 612 may be similar to operation 510 in FIG. 5.

Operations 608-610 and operations 608-612 may repeat as required or desired. For example, a fourth set of measurements for a fourth inhalation phase may be received and the set of prior measurements may be updated to include one or more of the first set of measurements, the second set of measurements, the third set of measurements, and the fourth set of measurements. One or more of the first set of measurements, the second set of measurements, or the third set of measurements may be removed from the set of prior measurements when updating with the fourth set of measurements. This may continue as additional inhalation phases pass over time. The model parameters, the lung resistance, and the lung compliance may be re-estimated after updating the set of prior measurements.

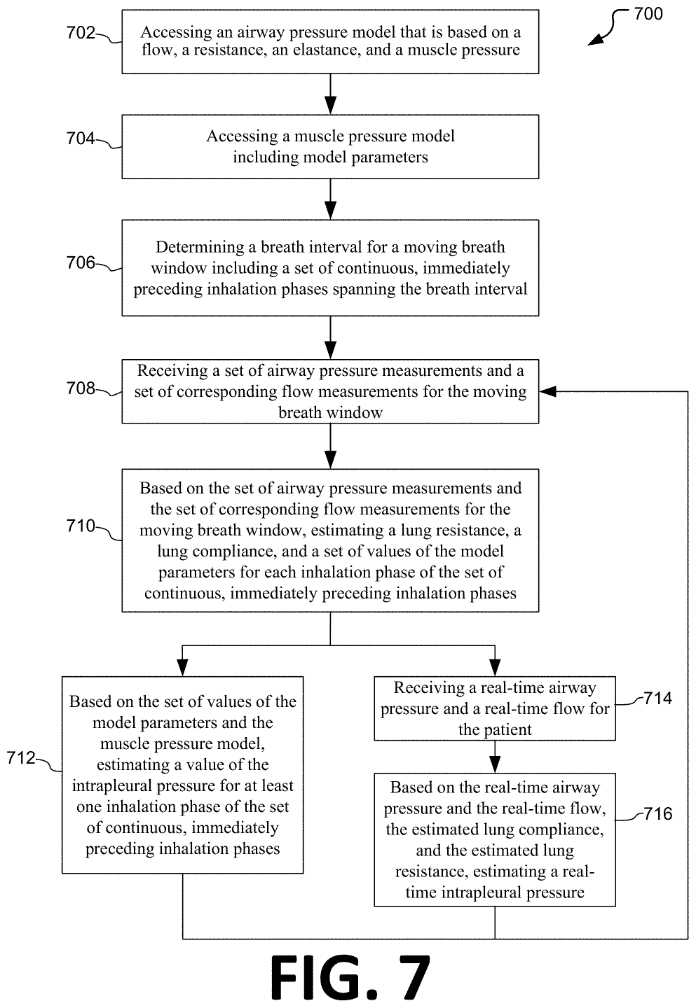

FIG. 7 is an example method 700 for non-invasive estimation of intrapleural pressure based on a moving window of prior breaths. Similar to method 600 in FIG. 6, method 700 utilizes a specific subset of past ventilation data, namely ventilation data that falls within a moving breath window. Additionally, method 700 shows a plurality of uses for values estimated using the past ventilation data. At operation 702, an airway pressure model that is based on a lung flow, a lung resistance, a lung elastance, and an intrapleural pressure is accessed. The airway pressure model may be based on an equation of motion (e.g., the equation of motion of Eqn. 1 or Eqn. 12), such as the airway pressure model of Eqn. 2 or Eqn. 13. Alternatively, the airway pressure model may be any other relationship between the airway pressure and the intrapleural pressure.

At operation 704, a muscle pressure model for estimating the intrapleural pressure is accessed, the muscle pressure model including model parameters. The muscle pressure model may be any model of any degree or any basis that is adjustable to uniquely model the intrapleural pressure for the inhalation phase of each breath, as further described herein.

At operation 706, a breath interval for a moving breath window is determined. In this example, the moving breath window includes a set of continuous, immediately preceding inhalation phases spanning the breath interval for a patient. The breath interval is the quantity of breaths included in the moving breath window at any given time. The breath interval may be selected by a clinician or determined by the ventilator. In an example, the breath interval is between two to ten breaths. Determination of the breath interval may be based on a confidence interval for an estimated lung resistance or lung compliance, accuracy of a previously estimated lung resistance or lung compliance, the amount of computational time or energy required to estimate one or more adjustable parameters (e.g., model parameters for the muscle pressure model, the lung resistance, or the lung compliance), the quantity or frequency of measurements of ventilation data, etc. For example, the breath interval may be increased if a previously estimated lung resistance is not accurate or has a wide confidence interval. Alternatively, the breath interval may be decreased if estimations of adjustable parameters take too long or are too computationally intensive or if there is a large quantity of measured ventilation data used in estimations.

The moving breath window includes a set of continuous, immediately preceding inhalation phases spanning the breath interval. As further described herein, if the amount of continuous, immediately preceding inhalation phases is less than the breath interval, then all of the available prior breaths may be included in the moving breath window. After the available prior breaths equal or exceed the breath interval, the quantity of breaths in the moving breath window equals the breath interval. For example, if the breath interval is three breaths (with three corresponding inhalation phases), then for breaths 1, 2, 3, 4, and 5 (where breath 1 is the first breath with available ventilation data) the moving breath window includes the following quantity of breaths after each respective breath: 1 (breath 1), 2 (breaths 1 and 2), 3 (breaths 1, 2, and 3), 3 (breaths 2, 3, and 4), and 3 (breaths 3, 4, and 5).

At operation 708, a set of airway pressure measurements and a set of corresponding flow measurements for the moving breath window are received. Each measurement of the set of airway pressure measurements and the set of corresponding flow measurements may be associated with a time at which the measurement was taken and a breath count for the breath during which the measurement was taken. Thus, each measurement is correlated with a specific time during a specific inhalation phase of the moving breath window. The set of airway pressure measurements and set of corresponding flow measurements updates to include measurements taken during the moving breath window. Continuing the example above with breaths 1-5, for the moving breath window after breath 4 (including breaths 2, 3, and 4) the set of airway pressure measurements and set of corresponding flow measurements includes measurements taken during the inhalation phases of breaths 2, 3, and 4, but not breath 1. For the moving breath window after breath 5 (including breaths 3, 4, and 5) the set of airway pressure measurements and set of corresponding flow measurements includes measurements taken during the inhalation phases of breaths 3, 4, and 5, but not breaths 1 or 2.

At operation 710, based on the set of airway pressure measurements and the set of corresponding flow measurements for the moving breath window, a lung resistance, a lung compliance, and a set of values of the model parameters for each inhalation phase of the set of continuous, immediately preceding inhalation phases are estimated. As further described herein, the model parameters are associated with the muscle pressure model such that the muscle pressure model is adjustable to uniquely model different inhalation phases for which ventilation data is measured. Thus, as the breaths included in the moving breath window change, the set of airway pressure measurements and set of corresponding flow measurements change and the model parameters change. Continuing the example above, after breath 4 (including breaths 2, 3, and 4) model parameters are estimated for the inhalation phase of breaths 2, 3, and 4. After breath 5 (including breaths 3, 4, and 5) model parameters are estimated for the inhalation phase of breaths 3, 4, and 5 (re-estimating the model parameters associated with breaths 3 and 4). A common lung resistance and common lung compliance is estimated each breath in moving breath window. For example, after breath 4, one lung resistance and one lung compliance are estimated. After breath 5, the lung resistance and the lung compliance are re-estimated. This operation 710 may be similar to operation 612 in FIG. 6.

Flow may then proceed to operation 712 and/or operations 714-716. At operation 712, based on the set of values of the model parameters and the muscle pressure model, value(s) of the intrapleural pressure for inhalation phase(s) of the set of continuous, immediately preceding inhalation phases is estimated. The estimated model parameters for each breath of the moving breath window adjust the muscle pressure model to be unique to each breath in the moving breath window. Thus, each inhalation phase associated with a set of model parameters may be modeled by the muscle pressure model by modifying the muscle pressure model based on the set of model parameters specific to that inhalation phase. As an alternative to a complete model of one or more inhalation phases, the intrapleural pressure at any given time during an inhalation phase of the moving breath window may be estimated using the muscle pressure model with the associated model parameters.

Additionally or alternatively, flow may proceed to operations 714-716. At operation 714, a real-time airway pressure and a real-time flow for the patient are received. At operation 716, based on the real-time airway pressure and the real-time flow, the estimated lung compliance, and the estimated lung resistance, a real-time muscle pressure or intrapleural pressure is estimated. As described at operation 702, the airway pressure model establishes a relationship between the airway pressure and the intrapleural pressure. Based on the airway pressure model, a proxy for the intrapleural pressure is estimated from the measured real-time airway pressure and other known or estimated parameters, such as the lung resistance and lung compliance (estimated at operation 710), the real-time flow, a real-time volume (as may be based on the real-time flow), PEEP, a leak flow, etc. These operations 714-716 may be similar to operations 408-410 in FIG. 4.

Operations 708-716 may repeat as required or desired. For example, as ventilation data is measured for a new breath, the moving breath window moves and the set of airway pressure measurements and set of corresponding flow measurements changes accordingly. Continuing the example above, after new breath 6, the moving breath window includes breaths 4, 5, and 6, and the set of airway pressure measurements and set of corresponding flow measurements includes ventilation data measured during the respective inhalation phases of breaths 4, 5, and 6, but not breaths prior to breath 4.

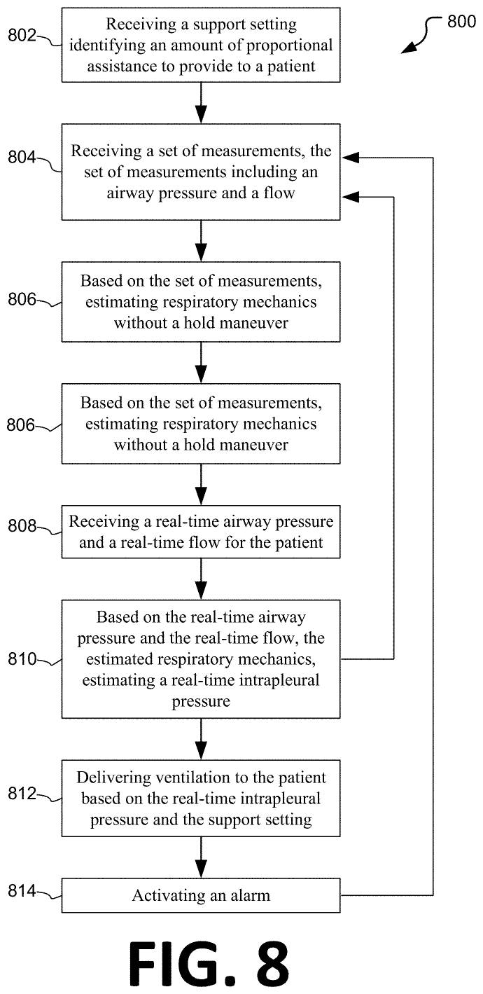

FIG. 8 is an example method 800 for non-invasive estimation of intrapleural pressure as implemented with a support setting. Method 800 may implement aspects of other methods described herein in the context of a support setting provided during ventilation. For example, method 800 relates to delivering ventilation in modes of ventilation with support settings that would otherwise require a maneuver that is not accurate in the presence of leak. One ventilation mode that traditionally requires a maneuver to determine lung resistance and lung compliance is proportional assist ventilation (PAV). PAV refers to a type of ventilation in which the ventilator acts as an inspiratory amplifier that provides pressure support based on the patient's muscle pressure.

At operation 802, a support setting identifying an amount of proportional assistance to provide to the patient is received. The support setting, or the “degree of amplification” is set by an operator, for example as a percentage based on the muscle pressure. In one implementation of PAV, the ventilator may continuously monitor the patient's instantaneous inspiratory flow, Q, and instantaneous inspired lung volume, ∫Q, which are indicators of the patient's muscle pressure. These signals, together with ongoing estimates of the lung compliance and lung resistance, allow the ventilator to compute the instantaneous pressure at a point in the ventilation circuit that assists the patient's inspiratory muscles to the degree selected by the operator as the support setting. With the present technology, PAV is able to utilize an improved estimate of muscle pressure, lung resistance, and/or lung compliance, as discussed herein.

PAV relies on certain physiological principles. The act of inspiration requires the patient's inspiratory muscles to develop a pressure gradient between the mouth and the alveoli sufficient to draw in breathing gas and inflate the lungs. Some of this pressure gradient is dissipated as gas travels through the artificial airway and the patient's conducting airways, and some of the pressure gradient is dissipated in the inflation of the lungs and thorax. Each element of pressure dissipation is characterized by a measurable property: the resistance of the artificial and patient airways, and the compliance (or elastance of the lung and thorax.

The ventilator providing PAV uses specific information, including resistance of the artificial airway, resistance of the patient's airways, lung compliance, instantaneous inspiratory flow and inspired lung volume, and the support setting to compute the instantaneous pressure to be applied at the wye. PAV begins inspiratory assist when flow generated by the patient's inspiratory muscles is detected. If the patient ceases inspiration, the assist also ceases. Once inspiratory flow begins, the ventilator monitors instantaneous flow and volume and applies the pressure calculated to deliver a proportion (determined by the support setting) of the total demand, Pₜₒₜ, as determined in part by the estimated patient's muscle pressure. In Tube Compensation (TC) ventilation, the ventilator monitors instantaneous flow and volume and applies the pressure calculated to overcome a proportion (determined by the support setting) of the pressure losses dissipated across the resistance of the artificial airways (e.g., endotracheal tube).

Because PAV relies on the patient's respiratory mechanics and muscle pressure, a more accurate determination of respiratory mechanics and muscle pressure may improve performance of the ventilator when providing PAV. If an estimate of the patient's muscle pressure is inaccurate (as may be due to an inaccurate lung resistance or lung compliance), then the calculated total pressure demand, Pₜₒₜ, is inaccurate and the proportional assist (as a proportion of the total demand as set in a support setting) is also inaccurate. If the proportional assist is too low (e.g., when the estimated muscle pressure, Pₘᵤₛ, is too low causing the total pressure, Pₜₒₜ, to be too low) the patient muscles may exert too much effort resulting in fatigue from over-loading. If the proportional assist is too high (e.g., when the estimated muscle pressure, Pₘᵤₛ, is too high causing the total pressure, Pₜₒₜ, to be too high) then the patient muscles may exert too little effort leading to muscle atrophy from non-use. Thus, an accurate estimation of muscle pressure and intrapleural pressure is important to prevent lung injury and enhance patient comfort.

Unlike previous technology relating to PAV, which estimates lung resistance during exhalation, but supports the patient during inhalation, aspects described herein estimate lung resistance during inhalation. Estimation of respiratory mechanics during inhalation to support the patient during inhalation may reduce over-supporting of the patient caused by an over-estimation of lung resistance during exhalation. Additionally, maintaining a desired intrapleural pressure may provide the patient with several benefits, such as prevention of muscle atrophy non-use and prevention of muscle fatigue from over-loading. Further, controlling and/or adjusting ventilation based on a patient's muscle pressure may help maintain a desired treatment metric range. Moreover, aspects of the present technology shown at least in method 800, among others, differ from previous technology by determining respiratory mechanics without using a maneuver.

At operation 804, a set of measurements is received, the set of measurements including airway pressure measurement(s) and flow measurement(s). The set of measurements is taken during an inhalation phase of a prior breath. The set of measurements may include measurements taken during two or more inhalation phases of respective two or more prior breaths, as further described herein. Each measurement of the set of measurements may be associated with a time at which the measurements was taken and/or the breath during which the measurement was taken.

At operation 806, a lung compliance and a lung resistance are estimated without a hold maneuver, based on the set of measurements. Methods and systems of estimating lung such compliance and lung resistance values are further described herein. For example, a non-invasive muscle pressure model with adjustable model parameters may be estimated for one or more breaths along with the lung compliance and lung resistance (e.g., operations 314, 406, 510, 612, 710).

At operation 808, an alarm limit is determined. The alarm limit may be based on the respiratory mechanics (e.g., lung compliance, lung resistance, or lung elastance) estimated at operation 806, the set of measurements, or an intrapleural pressure (as may be determined based on the model parameters or an equation of motion and the estimated respiratory mechanics). As further described herein, the drive pressure, P_{drive}, is ideally the maximum transpulmonary pressure, P_{L}, over time. An alarm setting may be based on if the real-time transpulmonary pressure, P_{L} (i.e., the difference between the real-time airway pressure and the real-time estimation of intrapleural pressure) exceeds the drive pressure, P_{drive}, based on prior ventilation data. Alarming based on if the real-time transpulmonary pressure exceeds the drive pressure may be preferable to alarming based on airway pressure.

At operation 810, a real-time airway pressure and a real-time flow for the patient are received. At operation 812, based on the real-time airway pressure and the real-time flow, the estimated lung compliance, and the estimated lung resistance, a real-time muscle pressure or intrapleural pressure is estimated (or a proxy for muscle pressure or a proxy for intrapleural pressure). A respiratory mechanics model may be referenced that establishes a relationship between known or estimated parameters (e.g., the real-time airway pressure, the real-time flow, the lung compliance, and the lung resistance) and the intrapleural pressure. Based on the respiratory mechanics model and the known or estimated parameters, an intrapleural pressure is estimated. These operations 810-812 may be similar to operations 408-410 in FIGS. 4 and 714-716 in FIG. 7.

At operation 814, ventilation is delivered to the patient based on the real-time muscle pressure (or proxy for the muscle pressure) or real-time intrapleural pressure (or proxy for the intrapleural pressure) and the support setting. This operation 814 may be similar to operation 412 in FIG. 4. As described herein, the proportional assist for PAV is provided based on a proportion of the total pressure, Pₜₒₜ, which is based on the muscle pressure, Pₘᵤₛ. Thus, with a real-time estimation of Pₘᵤₛ, an amount of support may be determined in real time. Although method 800 describes using the estimated values of lung resistance and lung compliance based on a non-invasive estimation of intrapleural pressure for implementation with PAV, estimated respiratory mechanics may also be implemented for mandatory breaths or any other mode of ventilation.

At operation 816, which may occur concurrently with operation 814, an alarm is activated. An alarm is activated based on a determination that an alarm threshold is met or exceeded. For example, if the alarm limit or alarm setting set at operation 808 is exceeded, then an alarm may sound or be displayed. The alarm may be a visual indicator and/or an audible indicator. The alarm may be displayed on the display of the ventilator and/or provided by a speaker of the ventilator.

Operations 804-816 may repeat as required or desired. For example, ventilation delivered to the patient may be adjusted based on changes in the set of real-time measurements, the real-time muscle pressure or real-time intrapleural pressure, the estimated lung compliance, the estimated lung resistance, and the set of measurements.

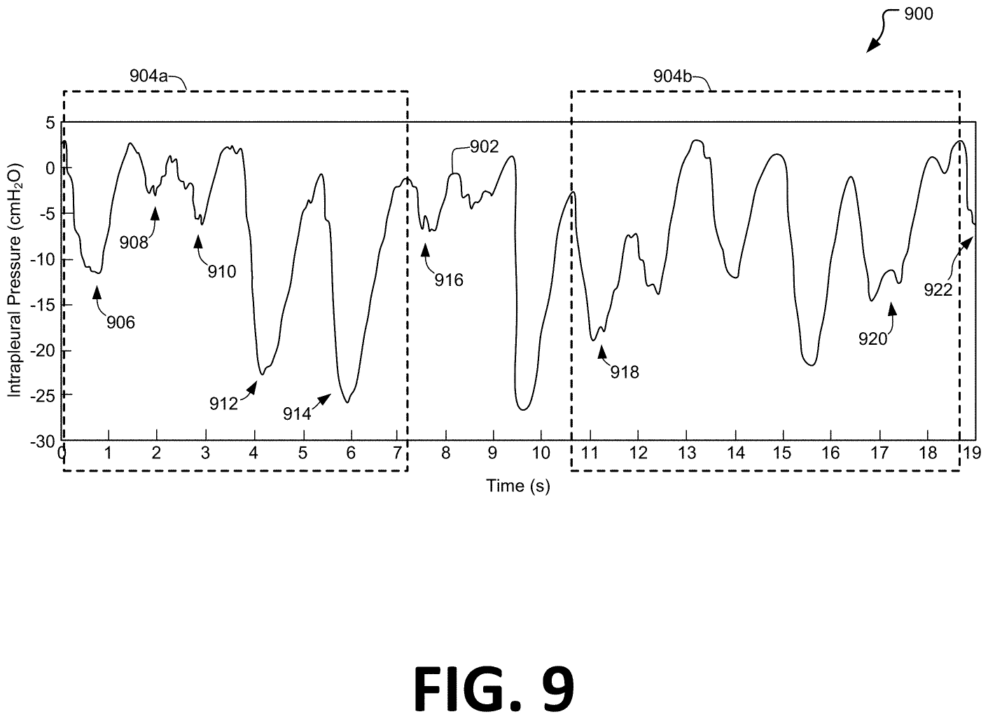

FIG. 9 is a graphical example 900 of intrapleural pressure 902 over time for a patient with acute respiratory distress syndrome (ARDS). The plot depicted in FIG. 9 includes a first moving breath window 904a and a second moving breath window 904b. The intrapleural pressure 902 shown in FIG. 9 was generated by simulation with a mechanical lung to provide an example of how the intrapleural pressure 902 may vary from breath to breath. The intrapleural pressure 902 shown was acquired over a plurality of breaths, including first breath 906, second breath 908, third breath 910, fourth breath 912, fifth breath 914, sixth breath 916, ninth breath 918, thirteenth breath 920, and fourteenth breath 922. The slope of the intrapleural pressure 902 trends negative over the inhalation phase of a breath, with the slope trending positive during exhalation. Thus, the intrapleural pressure 902 for a breath trends as concave up with a local minimum for each breath when the breath transitions from inhalation to exhalation. As the intrapleural pressure 902 becomes more negative, the lungs expand into the intrapleural space, forcing air into the lungs during inhalation. In contrast, as the intrapleural pressure 902 becomes less negative, the lung volume decreases, forcing air out of the lungs during exhalation. Thus, at the local minimum of intrapleural pressure 902 during a breath, when a breath is transitioning from inhalation to exhalation, the lung volume is maximized for the breath.

As further described herein, the moving breath windows (e.g., first moving breath window 904a and second moving breath window 904b) are associated with a breath interval over which the moving breath windows span. As shown in FIG. 9, the breath interval is five breaths (i.e., the moving breath windows span over five breaths). For example, the first moving breath window 904a includes five breaths (first breath 906, second breath 908, third breath 910, fourth breath 912, and fifth breath 914) spanning approximately 7.2 seconds. The second moving breath window 904b includes five breaths (the ninth breath 918 through the thirteenth breath 920) spanning approximately 8.1 seconds.

The moving breath window may include the breaths immediately preceding the current (or real time) breath. If the quantity of available past breaths is less than the breath interval for the moving breath window, then the moving breath window includes all available past breaths. Assuming that the breath interval for the moving breath window is five breaths, when the current breath is the second breath 908, the moving breath window includes the first breath 906. When the current breath is the third breath 910, the moving breath window includes the first breath 906 and the second breath 908. When the current breath is the fourth breath 912, the moving breath window includes the first breath 906, the second breath 908, and the third breath 910. When the current breath is the fifth breath 914, the moving breath window includes the first breath 906, the second breath 908, the third breath 910, and the fourth breath 912. When the current breath is the sixth breath 916, the quantity of available prior breaths equals the breath interval of five breaths. Thus, in the example shown in FIG. 9, the moving breath window for the sixth breath and each subsequent breath includes five breaths. For example, when the sixth breath 916 is occurring in real time, the moving breath window is the first moving breath window 904a. When the fourteenth breath 922 is occurring in real time, the moving breath window is the second moving breath window 904b.

Additionally, each breath of a patient is associated with a unique muscle pressure profile and unique intrapleural pressure profile. For example, the pressure profile of the first breath 906 is different from any other breath. Thus, when modeling the intrapleural pressure for a patient, the model should be adjustable to uniquely conform to the profile for each breath. As described herein, a common model to estimate each breath may be uniquely adjusted for each breath by changing model parameters (e.g., a muscle pressure model with adjustable model parameters described herein).

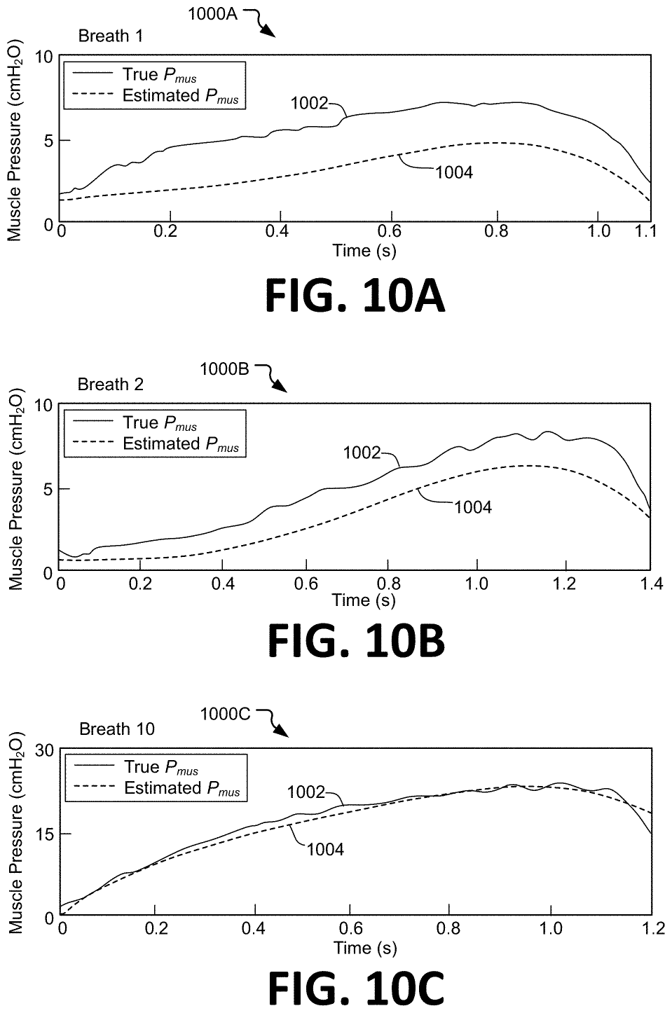

FIGS. 10A-C are graphical examples 1000A-C of true muscle pressure 1002 (or true Pₘᵤₛ 1002) and estimated muscle pressure 1004 (or estimated Pₘᵤₛ 1004), as estimated using the techniques described herein. FIG. 10A shows a graphical example 1000A of the true muscle pressure 1002 and the estimated muscle pressure 1004 for a first breath of a patient, FIG. 10B shows a graphical example 1000B of the true muscle pressure 1002 and the estimated muscle pressure 1004 for a second breath of the patient, and FIG. 10C shows a graphical example 1000C of the true muscle pressure 1002 and the estimated muscle pressure 1004 for a tenth breath of the patient. Based on ventilation data from prior breaths, model parameters for a muscle pressure model (as described above) may be adjusted to create a breath profile of the estimated muscle pressure (e.g., estimated muscle pressure 1004) for the inhalation phase of each breath. Various parameters for each estimated muscle pressure profile may be monitored, such as the peak, or maximum, estimated muscle pressure 1004.

In FIGS. 10A-C, the true muscle pressure 1002 is calculated based on the equation of motion of Eqn. 1 from measured airway pressure, measured flow, and the known a lung compliance and known lung resistance of a simulated patient lung (or mechanical lung) (e.g., in this case, C=40 mL/cmH₂O and R=20 cmH₂O/(L/s)), under pressure support ventilation. The estimated muscle pressure 1004 is based on techniques described herein, with a moving breath window of five breaths. Examples of the parameter vector, Oₙ, for the n^{th} breath (e.g., in graphical example 1000A, n=1; in graphical example 1000A, n=2; in graphical example 1000A, n=10), with the muscle pressure model having five model parameters (α_{n,0}, α_{n,1}, α_{n,2}, α_{n,3}, α_{n,4}) are represented as follows:

θ₁=[R₁E₁α_{1,0}α_{1,1}α_{1,2}α_{1,3}α_{1,4}α_{1,5}] (Eqn. 19)

θ₂=[R₁₋₂E₁₋₂α_{1,0}α_{1,1}α_{1,2}α_{1,3}α_{1,4}α_{1,5}α_{2,0}α_{2,1}α_{2,2}α_{2,3}α_{2,4}α_{2,5}] (Eqn. 20)

θ₁₀=[R₆₋₁₀E₆₋₁₀α_{1,0}α_{1,1}α_{1,2}α_{1,3}α_{1,4}α_{1,5}α_{7,0}α_{7,1}α_{7,2}α_{7,3}α_{7,4}α_{7,5}α_{8,0}α_{8,1}α_{8,2}α_{8,3}α₈₄α_{8,5}α_{9,0}α_{9,1}α_{9,2}α_{9,3}α_{9,4}α_{9,5}α_{10,0}α_{10,1}α_{10,2}α_{10,3}α_{10,4}α_{10,5}] (Eqn. 21)

where Rₙ₋ₘ and Eₙ₋ₘ represent the lung resistance and the lung elastance, respectively, common to breaths n through m. In this case, the initial values for the parameters of the parameter vector were taken from the prior estimate.

Table 1, below, shows the peak of the true muscle pressure 1002 and the peak of the estimated muscle pressure 1004, as well as the estimated lung resistance and estimated lung compliance for breaths one, two, and ten. As detailed above, the true lung resistance and lung compliance for the simulation shown in FIGS. 10A-C are C=40 mL/cmH₂O and R=20 cmH₂O/(L/s).

TABLE 1

| | | | |
|---|---|---|---|
| Breath, n | 1 | 2 | 10 |
| Peak True Muscle Pressure (cmH₂O) | 7.4 | 8.3 | 24 |
| Peak Estimated Muscle Pressure (cmH₂O) | 4.8 | 6.2 | 23.2 |
| Percent Pressure Difference | 35.1% | 25.3% | 3.3% |
| Estimated Lung Resistance (cmH₂O/(L/s)) | 15.4 | 17.9 | 18.5 |
| Percent Resistance Difference | 23.0% | 13.6% | 8.4% |
| Estimated Lung Compliance (mL/cmH₂O) | 43.3 | 44.6 | 37.8 |
| Percent Compliance Difference | 8.3% | 10.6% | 4.9% |

The percent differences are calculated by taking the absolute value of the difference between the true value and the estimated value, dividing by the true value, and multiplying by 100%. As shown in Table 1 and FIGS. 10A-C, with more breath data (e.g., the five breaths included in breath ten), the estimates for each parameter of the parameter vector become more accurate. For instance, as shown in Table 1, the percent differences are reduced by breath ten. With respect to the plots depicted in FIGS. 10A-C, the profile of the estimated muscle pressure becomes much closer to that of the true muscle pressure by breath 10. Accuracy may also improve, or more quickly improve, with better initial values for the parameters of the parameter vector, as well as improving with additional ventilation data (e.g., five breaths for breath 10, versus one breath for breath one and two breaths for breath two).

Although the present disclosure discusses the implementation of these techniques in the context of a ventilator capable of performing non-invasive estimation of intrapleural pressure, the techniques introduced above may be implemented for a variety of medical devices or devices utilizing flow sensors. A person of skill in the art will understand that the technology described in the context of a medical ventilator for human patients may be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for delivering ventilation, the method comprising:
receiving a support setting identifying an amount of proportional assistance to provide to the patient;
receiving a set of measurements, the set of measurements including at least one airway pressure and at least one flow measurement;
based on the set of measurements, estimating a lung compliance and a lung resistance, without using a hold maneuver;
receiving a real-time airway pressure and a real-time flow for a current breath;
based on the real-time airway pressure, the real-time flow, the estimated lung compliance, and the estimated lung resistance, estimating at least one of a real-time intrapleural pressure or a real-time muscle pressure; and
delivering ventilation to the patient based on the support setting and the at least one of the real-time intrapleural pressure or the real-time muscle pressure.

2. The method of claim 1, wherein the set of measurements includes a first subset of measurements from a first inhalation phase of a first breath and a second subset of measurements from a second inhalation phase of a second breath, wherein the first breath and the second breath occur prior to the current breath.

3. The method of claim 2, wherein the estimated lung resistance and the estimated lung compliance are common to the first breath and the second breath.

4. The method of claim 1, wherein the at least one flow measurement of the set of measurements is leak-compensated.

5. The method of claim 4, the method further comprising:
estimating a real-time leak flow, wherein the at least one of the real-time intrapleural pressure or the real-time muscle pressure is further based on the real-time leak flow.

6. The method of claim 1, wherein ventilation is delivered according to a pressure assist ventilation (PAV) mode.

7. The method of claim 1, the method further comprising:
identifying a muscle pressure model with a set of model parameters; and
based on the set of measurements, estimating a set of values for the set of model parameters, wherein estimating the lung compliance and the lung resistance is further based on the set of values for the set of model parameters.

8. The method of claim 7, wherein the muscle pressure model is a fourth degree Bernstein basis polynomial.

9. A method for non-invasively estimating an intrapleural pressure of a patient, the method comprising:
identifying a muscle pressure model with a set of model parameters;
receiving a set of prior measurements for at least one prior breath, the set of prior measurements including at least one airway pressure and at least one flow measurement;
based on the set of prior measurements, estimating a lung compliance, a lung resistance, and at least one set of values for the set of model parameters;
receiving a set of real-time measurements for a current breath, wherein the set of real-time measurements includes at least one airway pressure and at least one flow measurement;
based on the set of real-time measurements, the at least one set of values for the set of model parameters, the muscle pressure model, the estimated lung compliance, and the estimated lung resistance, estimating at least one of an intrapleural pressure or a muscle pressure in real time; and
delivering ventilation to the patient based on the at least one of the real-time intrapleural pressure or the real-time muscle pressure.

10. The method of claim 9, wherein the lung compliance, the lung resistance, and the at least one set of values for the set of model parameters are estimated by minimizing an error between the at least one airway pressure of the set of prior measurements and a modeled airway pressure based on the at least one flow measurement of the set of prior measurements.

11. The method of claim 10, wherein the modeled airway pressure is:
P_{aw}=R*Q+E*∫Qdt+PEEP−Pₘᵤₛ.

12. The method of claim 9, wherein the set of prior measurements is received for at least a first prior breath and a second prior breath, and wherein the at least one set of values for the set of model parameters includes a first value set for the first prior breath and a second value set for the second prior breath.

13. A method for noninvasive estimation of intrapleural pressure, the method comprising:
accessing a muscle pressure model;
receiving a first set of measurements for a first inhalation phase of a patient and a second set of measurements for a second inhalation phase of the patient, the first set of measurements and the second set of measurements including at least one airway pressure and at least one flow;
based on the first set of measurements and the second set of measurements, estimating a parameter set, the parameter set including a first set of model parameters of the muscle pressure model for the first inhalation phase, a second set of model parameters of the muscle pressure model for the second inhalation phase, and a lung resistance and a lung compliance common to the first inhalation phase and the second inhalation phase; and
based on the second set of measurements, generating at least one value of at least one of an intrapleural pressure or a muscle pressure of the patient for the second inhalation phase.

14. The method of claim 13, wherein the at least one value for the second inhalation phase is a maximum value during the second inhalation phase.

15. The method of claim 13, the method further comprising:
accessing an airway pressure model, wherein the airway pressure model relates an airway pressure, a flow, a resistance, an elastance, and the muscle pressure, wherein the at least one value is generated based on the airway pressure model.

16. The method of claim 15, wherein the parameter set is estimated by minimizing an error between the airway pressure model and the at least one airway pressure for the first set of measurements and the second set of measurements.

17. The method of claim 15, wherein generating the at least one value for the second inhalation phase is further based on the lung resistance and the lung compliance common to the first inhalation phase and the second inhalation phase and the airway pressure model.

18. The method of claim 13, wherein the muscle pressure model is fourth degree with a Bernstein basis polynomial.

19. The method of claim 13, wherein generating the at least one value for the second inhalation phase is further based on the muscle pressure model and the second set of model parameters.

20. The method of claim 13, the method further comprising:
based on the muscle pressure model, the second set of model parameters, and the second set of measurements, generating a first profile of at least one of the intrapleural pressure or the muscle pressure for the second inhalation phase; and
based on the muscle pressure model, the first set of model parameters, and the first set of measurements, generating a second profile for the at least one of the intrapleural pressure or the muscle pressure for the first inhalation phase.
